(19) United States
(12) Patent Application Publication
(10) Pub. No.: US 2026/0021294 A1
(43) Pub. Date: Jan. 22, 2026

(54) MANICKA ZONE OF PACING

(71) Applicant: Manicka Institute LLC, Woodbury, MN (US)

(72) Inventor: Yatheendhar D. Manicka, Woodbury, MN (US)

(21) Appl. No.: 19/277,233

(22) Filed: Jul. 22, 2025

Related U.S. Application Data
(60) Provisional application No. 63/674,205, filed on Jul. 22, 2024.

(51) Int. Cl. A61N 1/05; A61N 1/37; A61N 1/375

(52) U.S. Cl. CPC A61N 1/059; A61N 1/3702; A61N 1/37512

(57) ABSTRACT

A method of pacing a heart includes implanting a subcutaneously implantable device on a xiphoid process and/or a sternum of a patient. The subcutaneously implantable device includes a housing, a prong extending away from the housing, and a first electrode at a distal end of the prong. The method further includes contacting a Manicka Zone of Pacing on an anterior surface of the heart with the first electrode on the distal end of the prong. The Manicka Zone of Pacing is defined as an area of the heart extending 3 centimeters (1.1811 inches) laterally outward from either side of a left anterior descending artery of the heart. A pacing signal is delivered to the Manicka Zone of Pacing of the heart through the first electrode. A right ventricle and a left ventricle of the heart are synchronously paced with the pacing signal received in the Manicka Zone of Pacing.

### CROSS-REFERENCE TO RELATED APPLICATION(S)

This application claims priority to U.S. Provisional Application No. 63/674,205, filed on Jul. 22, 2024, and entitled “Manicka Zone of Pacing,” the disclosure of which is incorporated by reference in its entirety.

### BACKGROUND

The present disclosure relates to cardiac pacing, and in particular, to pacing the heart to improve synchrony.

Cardiac pacing can be used to treat patients with symptomatic bradyarrhythmia. In traditional cardiac pacing devices, a lead is extended through the vasculature and implanted in the right ventricular apical, referred to as right ventricular pacing. Right ventricular pacing has been the standard cardiac pacing approach for nearly six decades. However, right ventricular pacing is limited due to associated electrical and mechanical dyssynchrony. Namely, pre-excitation of the ventricular septum coupled with delayed activation of the left ventricular free wall produces dyssynchronous activation and less effective contraction of the heart. Clinically this can translate into pacing-induced cardiomyopathy in up to 20% of patients and an increased risk for heart failure hospitalization during long-term follow up.

Recently, biventricular pacing has evolved as an alternative pacing treatment. Biventricular pacing includes extending a first lead through the vasculature and implanting it in the right ventricular apical, extending a second lead through the vasculature and implanting it in the coronary sinus, and extending a third lead through the vasculature and implanting it in the right atrium. The lead that is positioned in the coronary sinus is positioned to provide electrical stimulation to the left ventricle. Biventricular pacing is aimed at synchronizing the pacing of the right ventricle and left ventricle to promote effective contraction of the heart. A challenge of biventricular pacing is that it requires three leads to be positioned through the patient's vasculature and implanted in the heart, requiring a more complicated surgical procedure to implant the device and increasing the risk of stroke. Further, some patients are not candidates for biventricular pacing, as their vasculature does not allow for the placement of all three leads.

Pacing of the bundle of His and pacing of the left bundle branch, parts of the native conduction pathway of the heart, are also alternatives to traditional right ventricular pacing that are aimed at synchronizing the contraction of the heart. Pacing of the bundle of His and pacing of the left bundle branch have demonstrated significant efficacy in correcting left bundle branch block and achieving cardiac resynchronization therapy. To pace the bundle of His and/or to pace the left bundle branch, a lead is extended through the patient's vasculature and implanted in the interventricular septum. For pacing the bundle of His, the lead pierces the septum from the right ventricle side and is screwed into the His bundle. For pacing the left bundle branch, the lead pierces the septum, and is advanced deep enough to reach left side of the interventricular septum wall and the left bundle branch.

Bundle branch pacing, specifically left bundle branch pacing, has shown promising results as an alternative to traditional biventricular pacing for cardiac resynchronization therapy. Recent clinical studies have shown that bundle branch pacing improves clinical outcomes, reduces heart failure hospitalization, improves cardiac function, and provides for higher response rates. Specifically, it has been shown that bundle branch pacing increases left ventricular ejection fraction compared to biventricular pacing, is associated with shortened QRS duration, and reduces left ventricular end-diastolic dimension. These studies indicate that bundle branch pacing, and specifically left bundle branch pacing, is an improved treatment for heart failure patients with an indication for cardiac resynchronization therapy.

### SUMMARY

A method of pacing a heart includes implanting a subcutaneously implantable device on a xiphoid process and/or a sternum of a patient. The subcutaneously implantable device includes a housing, a prong extending away from the housing, and a first electrode at a distal end of the prong. The method further includes contacting a Manicka Zone of Pacing on an anterior surface of the heart with the first electrode on the distal end of the prong. The Manicka Zone of Pacing is defined as an area of the heart extending 3 centimeters (1.1811 inches) laterally outward from either side of a left anterior descending artery of the heart. A pacing signal is delivered to the Manicka Zone of Pacing of the heart through the first electrode. A right ventricle and a left ventricle of the heart are synchronously paced with the pacing signal received in the Manicka Zone of Pacing.

A method of pacing a heart includes implanting a subcutaneously implantable device on a xiphoid process and/or a sternum of a patient. The subcutaneously implantable device includes a housing, a prong extending away from the housing, and a first electrode and a second electrode at a distal end of the prong. The method further includes contacting a Manicka Zone of Pacing of the heart with the first electrode and/or the second electrode on the distal end of the prong. The Manicka Zone of Pacing is defined as an area of the heart extending 3 centimeters (1.1811 inches) laterally outward from either side of a left anterior descending artery of the heart. The first electrode and/or the second electrode contact the heart near the base of the heart within the Manicka Zone of Pacing. A pacing signal is delivered to the Manicka Zone of Pacing of the heart through the first electrode and/or the second electrode. The right ventricle and a left ventricle of the heart are synchronously paced with the pacing signal received in the Manicka Zone of Pacing.

A subcutaneously implantable device includes a housing, a clip attached to a top side of the housing that is configured to anchor the device to a xiphoid process and/or a sternum, and a prong with a proximal end attached to the housing and a distal end extending away from the housing that is configured to be positioned at a Manicka Zone of Pacing of a heart. The Manicka Zone of Pacing is defined as an area of the heart extending 3 centimeters laterally outward from either side of a left anterior descending artery of the heart. The device further includes a first electrode on the distal end of the prong that is configured to contact the heart in the Manicka Zone of Pacing, and a second electrode on the distal end of the prong, wherein the second electrode can be configured to contact the heart in the Manicka Zone of Pacing. Circuitry in the housing is in electrical communication with the first electrode and the second electrode and is configured to deliver a pacing signal to the Manicka Zone of Pacing of the heart.

A method of pacing a heart includes implanting a pacing device in a patient. The pacing device includes a housing and a first electrode electrically coupled to the housing. The method further includes contacting a Manicka Zone of Pacing on an anterior surface of the heart with the first electrode. The Manicka Zone of Pacing is defined as an area of the heart extending 3 centimeters (1.1811 inches) laterally outward from either side of a left anterior descending artery of the heart. A pacing signal is delivered to the Manicka Zone of Pacing of the heart through the first electrode. A right ventricle and a left ventricle of the heart are synchronously paced with the pacing signal received in the Manicka Zone of Pacing.

A method of pacing a heart includes implanting a pacing device in a patient. The pacing device includes a housing, a first electrode electrically coupled to the housing, and a second electrode electrically coupled to the housing. The method further includes contacting a Manicka Zone of Pacing of the heart with the first electrode and/or the second electrode. The Manicka Zone of Pacing is defined as an area of the heart extending 3 centimeters (1.1811 inches) laterally outward from either side of a left anterior descending artery of the heart, and wherein the first electrode and/or second electrode contact the heart near the base of the heart within the Manicka Zone of Pacing. A pacing signal is delivered to the Manicka Zone of Pacing of the heart through the first electrode and/or the second electrode. A right ventricle and a left ventricle of the heart are synchronously paced with the pacing signal received in the Manicka Zone of Pacing.

### BRIEF DESCRIPTION OF THE DRAWINGS

FIG. 1A is an anterior oblique view of a heart.

FIG. 1B is a cross-sectional view of the heart.

FIG. 2A is a side view of a first embodiment of a subcutaneous device anchored to a structural body component.

FIG. 2B is a top perspective view of the first embodiment of the subcutaneous device.

FIG. 2C is a side view of the first embodiment of the subcutaneous device.

FIG. 2D is a perspective side view of the first embodiment of the subcutaneous device positioned on a xiphoid process and/or a sternum and showing a positioning of a prong on the heart.

FIG. 3A is a side view of a second embodiment of a subcutaneous device anchored to a structural body component.

FIG. 3B is a perspective view of the second embodiment of the subcutaneous device.

FIG. 3C is a side view of the second embodiment of the subcutaneous device.

FIG. 3D is a perspective view of the second embodiment of the subcutaneous device positioned on a xiphoid process and/or a sternum and showing a positioning of prongs on the heart.

FIG. 4A is a first oblique side view of a third embodiment of a subcutaneous device contacting the heart.

FIG. 4B is a second oblique side view of the third embodiment of the subcutaneous device contacting the heart.

FIG. 5A is a schematic depiction of the third embodiment of the subcutaneous device contacting the heart in a first position.

FIG. 5B is a schematic depiction of the third embodiment of the subcutaneous device contacting the heart in a second position.

FIG. 5C is a schematic depiction of the third embodiment of the subcutaneous device contacting the heart in a third position.

FIG. 6 is a perspective of a fourth embodiment of a subcutaneous device.

FIG. 7 is a schematic depiction of the fourth embodiment of the subcutaneous device contacting the heart in a first position.

FIG. 8A is an electrocardiogram graph showing a QRS complex for a normal, healthy heart.

FIG. 8B is a first electrocardiogram graph showing a QRS complex when pacing a Manicka Zone of Pacing.

FIG. 8C is a second electrocardiograph graph showing a QRS complex when pacing the Manicka Zone of Pacing.

### DETAILED DESCRIPTION

In general, the present disclosure relates to pacing the heart in the Manicka Zone of Pacing. The Manicka Zone of Pacing is defined as an area of the heart extending 3 centimeters (1.1811 inches) laterally outward from either side of a left anterior descending artery of the heart. The heart can be paced in an area of the heart extending longitudinally from a region superior to the base of the heart to the apex of the heart within the Manicka Zone of Pacing. The heart can be paced in an area of the heart near the base of the heart within the Manicka Zone of Pacing. The heart can be contacted in the Manicka Zona of Pacing on the pericardium or the epicardium of the heart.

Pacing the heart on one or both sides of the left anterior descending artery or in two or more locations on the same side and close to the left descending artery allows for synchronous pacing. Pacing in the Manicka Zone of Pacing improves patient outcomes compared to traditional cardiac pacing therapies, demonstrated by narrower QRS complexes compared to traditional cardiac pacing therapies. Further, pacing the heart in the Manicka Zone of Pacing includes, but is not limited to, pericardial and epicardial pacing, which can be done using a device that is subcutaneously implanted in the patient. Leads do not have to be extended through the vasculature to pace the heart in the Manicka Zone of Pacing.

In general, the present disclosure also relates to a subcutaneous device that can be implanted into a patient for synchronous pacing. The subcutaneous device includes a housing that contains the electrical circuitry of the subcutaneous device, a clip on a top side of the housing, and one or more prongs extending away from the housing. The clip is configured to attach and anchor the subcutaneous device onto a muscle, a bone, or tissue. The prong extends away from the housing and a distal end of the prong comes into contact with the Manicka Zone of Pacing remote from the subcutaneous device.

The subcutaneous device can be a pacemaker and/or an implantable cardioverter-defibrillator. A pacemaker and an implantable cardioverter-defibrillator can sense a patient's heart rate and provide a therapeutic electrical stimulation to the patient's heart if an abnormality is detected. A pacemaker will provide an electrical stimulation to the heart in response to an arrhythmia, such as bradycardia, tachycardia, atrial flutter, and atrial fibrillation. The electrical stimulation provided by a pacemaker will contract the heart muscles to regulate the heart rate of the patient. An implantable cardioverter-defibrillator will provide an electrical stimulation to the heart in response to ventricular fibrillation and ventricular tachycardia, both of which can lead to sudden cardiac death. An implantable cardioverter-defibrillator will provide cardioversion or defibrillation to the patient's heart. Cardioversion includes providing an electrical stimulation to the heart at a specific moment that is in synchrony with the cardiac cycle to restore the patient's heart rate. Cardioversion can be used to restore the patient's heart rate when ventricular tachycardia is detected. If ventricular fibrillation is detected, defibrillation is needed. Defibrillation includes providing a large electrical stimulation to the heart at an appropriate moment in the cardiac cycle to restore the patient's heart rate. An implantable cardioverter-defibrillator can also provide pacing to multiple chambers of a patient's heart.

The subcutaneous device described in this disclosure can, in some embodiments, be anchored to a patient's xiphoid process and/or a distal end of a patient's sternum. The xiphoid process is a process on the lower part of the sternum. At birth, the xiphoid process is a cartilaginous process. The xiphoid process ossifies over time, causing it to fuse to the sternum with a fibrous joint. The subcutaneous device can be anchored to the xiphoid process so that the housing of the subcutaneous device is positioned below the xiphoid process and sternum. In some patients, the xiphoid process is absent, small, narrow, or elongated. In such cases, the subcutaneous device can be attached directly to the distal end of the patient's sternum. When the subcutaneous device is anchored to the xiphoid process and/or sternum, the one or more prongs of the subcutaneous device extend into the anterior mediastinum.

The subcutaneous device can include a number of embodiments, including a single chamber pacemaker, a dual chamber pacemaker, a triple chamber pacemaker, an atrial defibrillator, a single-vector ventricular defibrillator, and a multi-vector ventricular defibrillator. These embodiments are included as examples and are not intended to be limiting. The subcutaneous device can have any suitable design and can be used for any suitable purpose in other embodiments. The features of each embodiment may be combined and/or substituted with features of any other embodiment, unless explicitly disclosed otherwise. Further, many of the embodiments can be used for multiple purposes. For example, a defibrillator device can also be used for monitoring and pacing.

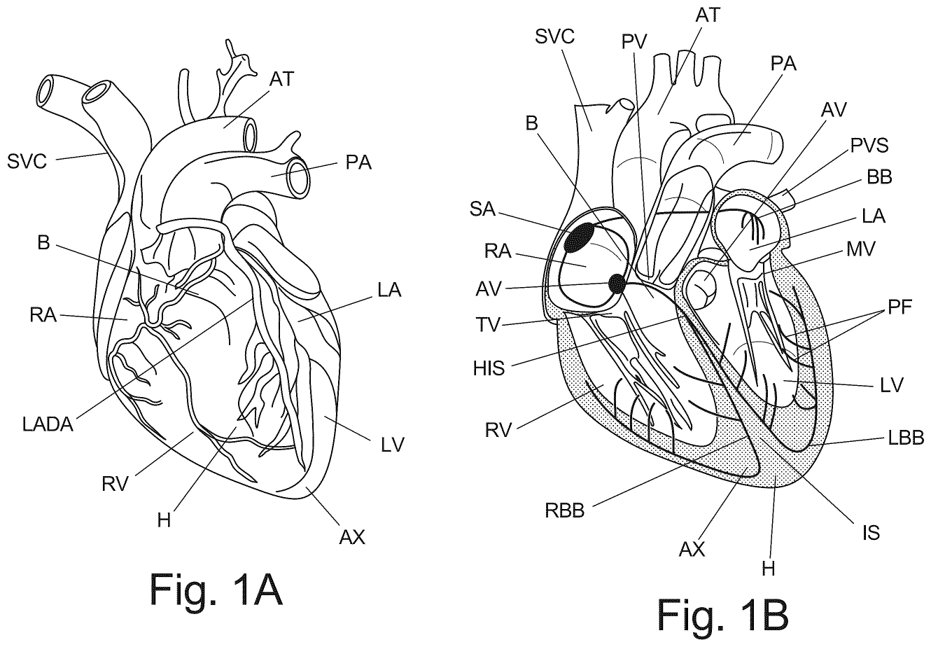

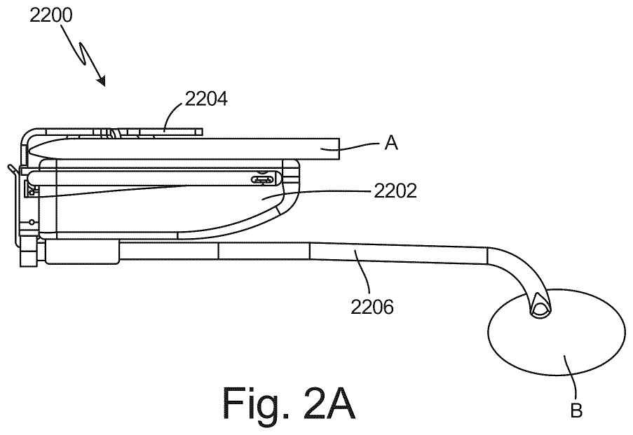

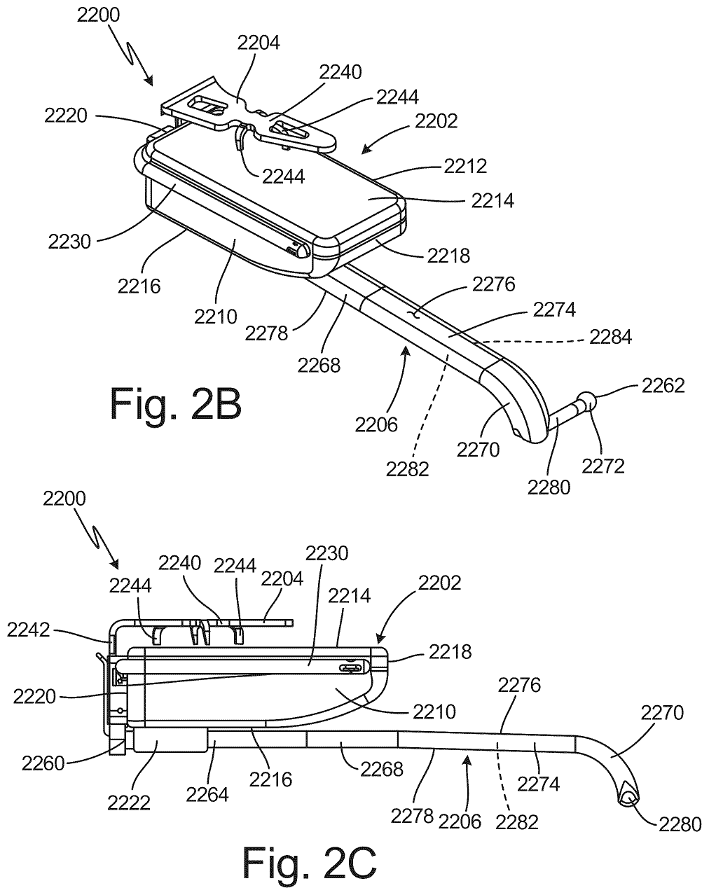

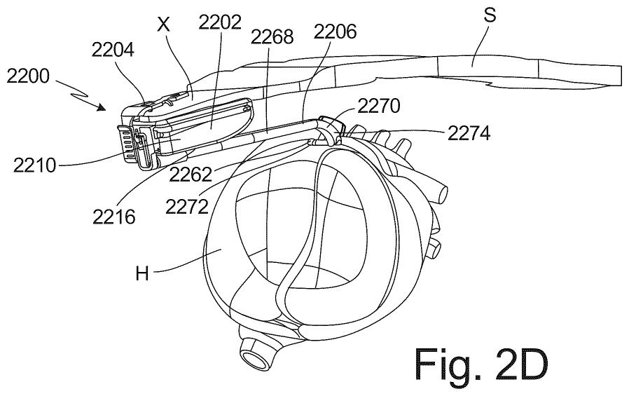

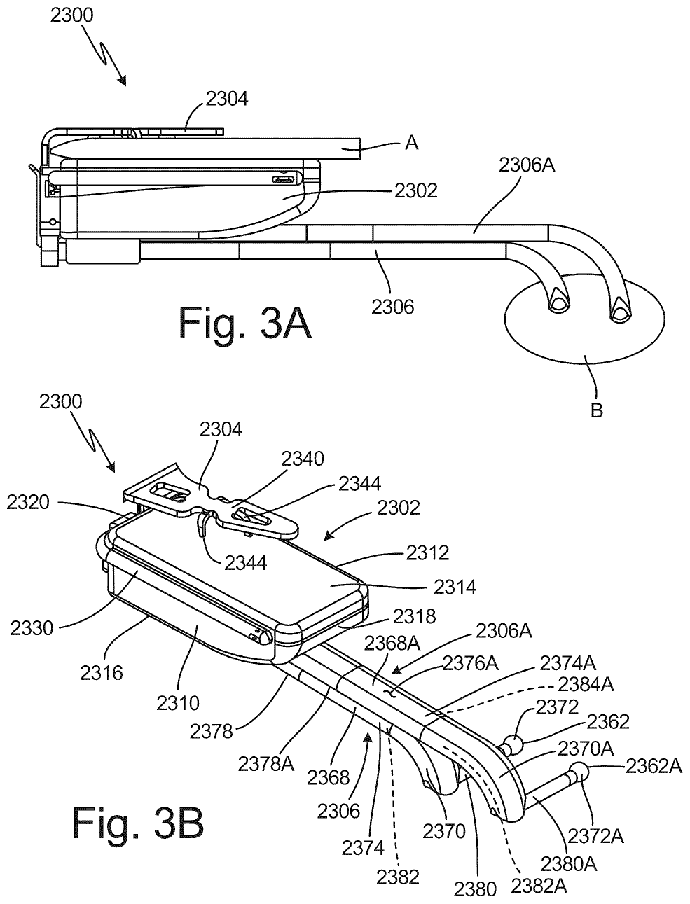

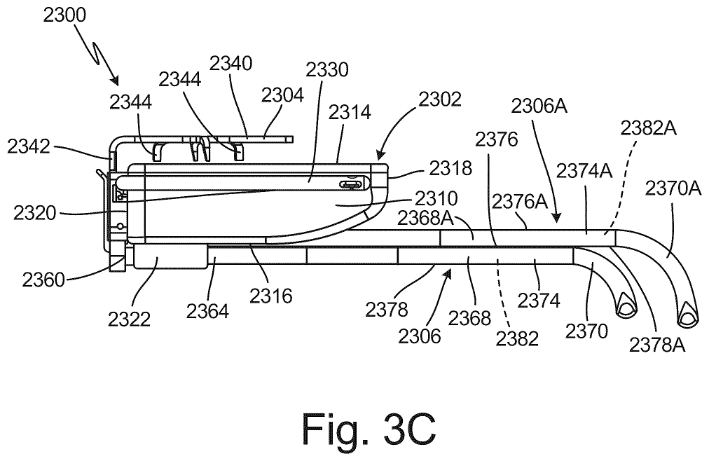

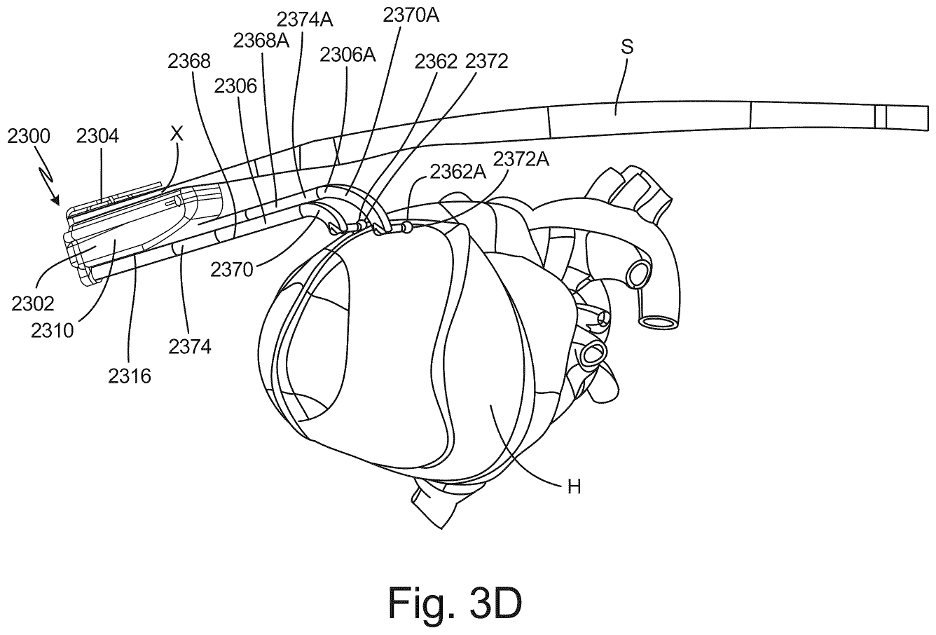

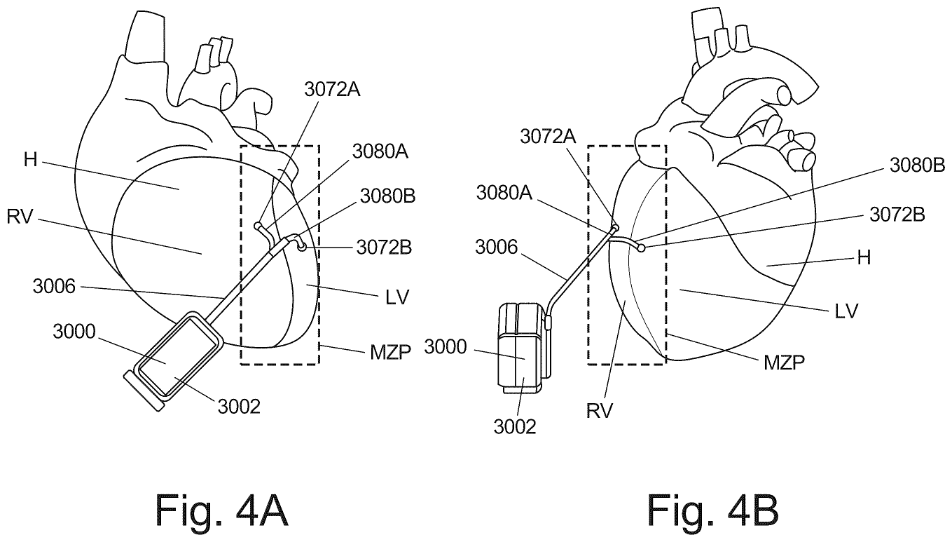

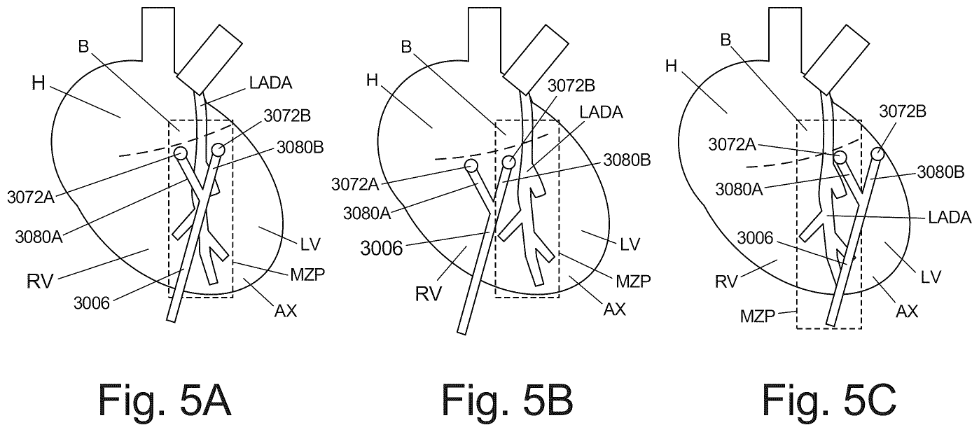

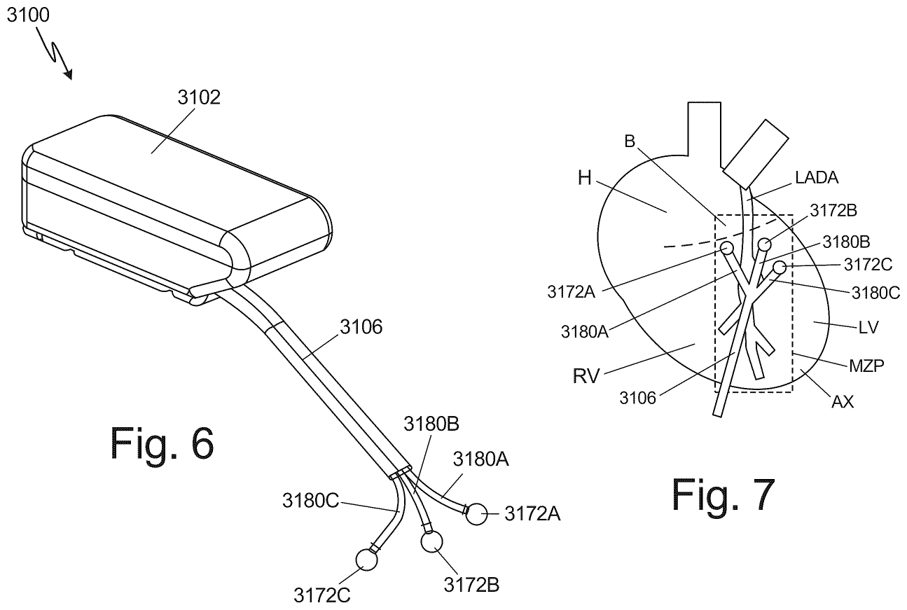

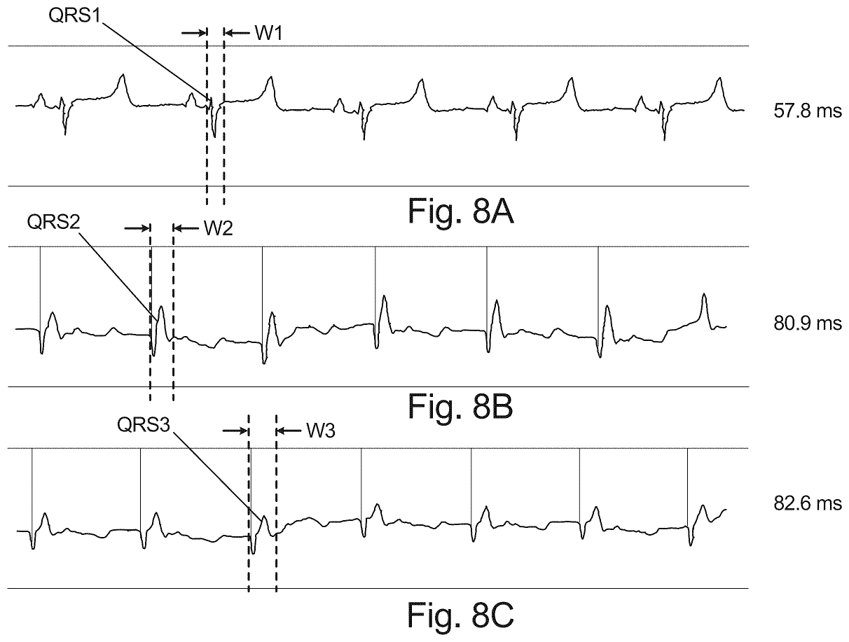

A heart circulation and native conduction pathway is described below with reference to FIGS. 1A-1B. Several examples of subcutaneous devices, including various configurations of prongs and electrodes, will be described below with reference to FIGS. 2A-3D. An example of a subcutaneous device having two electrodes interacting with the heart in the Manicka Zone of Pacing is described below with reference to FIGS. 4A-5C. An example of a subcutaneous device having three electrodes interacting with the heart in the Manicka Zone of Pacing is described below with reference to FIGS. 6-7. QRS complexes when pacing in the Manicka Zone of Pacing are described below with reference to FIGS. 8A-8C.

### Heart Circulation and Native Conduction Pathway

FIG. 1A is an anterior oblique view of heart H. FIG. 1B is a cross-sectional view of heart H. For clarity and ease of discussion, FIGS. 1A-1B will be described together. FIGS. 1A-1B show right atrium RA, right ventricle RV, left atrium LA, left ventricle LV, superior vena cava SVC, pulmonary artery PA, aorta AT, apex AX, and base B. FIG. 1A further shows left anterior descending artery LADA. FIG. 1B further shows tricuspid valve TV, pulmonary valve PV, mitral valve MV, aortic valve AV, interventricular septum IS, pulmonary veins PVS, sinoatrial node SA, atrioventricular node AV, Bachman's bundle BB, bundle of His HIS (also called the atrioventricular bundle), right bundle branch RBB, left bundle branch LBB, and Purkinje fibers PF.

Generally, heart H includes multiple vessels and chambers that facilitate blood circulation. Heart H delivers blood to and receives blood from other parts of the body through arteries and veins, respectively. Heart H includes four different chambers, including right atrium RA, right ventricle RV, left atrium LA, and left ventricle LV. Superior vena cava SVC and the inferior vena cava (not shown in FIGS. 1A-1B) deliver deoxygenated blood to right atrium RA. Super vena cava SVC is positioned superior to right atrium RA and extends in an inferior direction to connect to right atrium RA. Blood in right atrium RA can then move into right ventricle RV. Right ventricle RV is positioned adjacent to right atrium RA in an inferior direction. Tricuspid valve TV separates right atrium RA and right ventricle RV. Tricuspid valve TV opens and closes to facilitate blood movement between right atrium RA and right ventricle RV. Blood in right ventricle RV can move into pulmonary artery PA. Pulmonary artery PA is positioned superior to right ventricle RV and is separated from right ventricle RV with pulmonary valve PV. Pulmonary valve PV opens and closes to facilitate blood movement between right ventricle RV and pulmonary artery PA.

In operation, right atrium RA receives unoxygenated blood in part from superior vena cava SVC and the inferior vena cava (not shown in FIGS. 1A-1B). Right atrium RA contracts to pump the unoxygenated blood through tricuspid valve TV and into right ventricle RV. Right ventricle RV contracts to pump the unoxygenated blood from right ventricle RV into pulmonary artery PA through pulmonary valve PV. Pulmonary artery PA then splits into two vessels. The vessels lead to the lungs (not shown in FIGS. 1A-1B). Inside the lungs, the blood is reoxygenated after respiration.

Pulmonary veins PVS are connected laterally to left atrium LA and deliver oxygenated blood from the lungs into left atrium LA. The blood can move from left atrium LA into left ventricle LV. Left ventricle LV is positioned adjacent to left atrium LA in an inferior direction. Mitral valve MV separates left atrium LA and left ventricle LV. Mitral valve MV opens and closes to facilitate blood movement between left atrium LA and left ventricle LV. The blood in left ventricle LA can be pumped into aorta AT through aortic valve AV. Aorta AT is positioned superior to left ventricle LV. Aortic valve AV opens and closes to facilitate blood movement between left ventricle LV and aorta AT.

In operation, left atrium LA receives oxygenated blood from pulmonary veins PVS. Left atrium LA contracts to pump oxygenated blood through mitral valve MV and into left ventricle LV. Left ventricle LV contracts to pump the oxygenated blood through aortic valve AV and into aorta AT, which can deliver the oxygenated blood to the rest of the body.

Left atrium LA is laterally adjacent to right atrium RA, and left ventricle LV is laterally adjacent to right ventricle RV. Left atrium LA is separated from right atrium RV by an interatrial septum (not shown in FIGS. 1A-1B), and left ventricle LV and right ventricle RV are separated by interventricular septum IS. A most inferior region of left ventricle LV and right ventricle RV form apex AX of heart H. A superior region of left atrium LA and right atrium RA form base B of heart H.

Left anterior descending artery LADA is an artery that supplies blood to heart H. Left anterior descending artery LADA branches off the left main coronary artery and supplies blood to the left side of heart H. Left anterior descending artery LADA is the largest coronary artery. Left anterior descending artery LADA is on the anterior of heart H, is in the epicardium of heart H, and is in part aligned with interventricular septum IS. Left anterior descending artery LADA extends from base B to apex A of heart H. Left anterior descending artery LADA descends into the anterior interventricular groove of heart H and supplies blood to the anterior two-thirds of interventricular septum IS. Left anterior descending artery LADA and its branches, specifically the anterior septal perforating arteries, enter the myocardial of interventricular septum IS to provide this crucial blood supply. This makes left anterior descending artery LADA a vital artery for the function of heart H, as it supplies the anterior, lateral, and apical walls of left ventricle LV, most of right bundle branch RBB and left bundle branch LBB, and the anterior papillary muscle of the bicuspid valve.

As heart H contracts, blood circulation through heart H is facilitated by the native conductive system of heart H, including sinoatrial node SA, atrioventricular node AV, Bachmann's bundle BB, bundle of His HIS, right bundle branch RBB, left bundle branch LBB, and Purkinje fibers PF.

Sinoatrial node SA (also referred to as SA node) is located on an upper wall of right atrium RA near superior vena cava SVC. Sinoatrial node SA is coupled to Bachmann's bundle BB. Bachman's bundle BB extends laterally across right atrium RA and left atrium LA. Sinoatrial node SA is also coupled to atrioventricular node AV. Atrioventricular node AV (also referred to as AV node) is positioned within an area called the triangle of Koch, which is located in the central part of heart H between tricuspid valve TV, the coronary sinus, and the membranous portion of the interatrial septum. Atrioventricular node AV is coupled to bundle of His HIS, which is a bundle of fibers extending from atrioventricular node AV down interventricular septum IS. Bundle of His HIS diverges into right bundle branch RBB and left bundle branches LBB. Right bundle branch RBB and left bundle branches LBB each extend along a length of the interventricular septum IS to apex A of heart H. Right bundle branch RBB extends laterally across right ventricle RV along apex A of heart H, and left bundle branch LBB extends laterally across left ventricle LV along apex A of heart H. Each of right bundle branch RBB and left bundle branch LBB extend to Purkinje fibers PF. Purkinje fibers PF are branches of specialized nerve cells that send electrical signals to right ventricle RV and left ventricle LV. Purkinje fibers PF are in the subendocardial surface of the endocardium in right ventricle RV and left ventricle LV.

Sinoatrial node SA is the natural pacemaker of heart H and generates an electrical signal. The electrical signal propagates through left atrium and Bachmann's bundle BB, causing right atrium RA and left atrium LA to contract. The electrical signal also propagates through right atrium RA and reaches atrioventricular node AV. Atrioventricular node AV delays the electrical signal by a fraction of a second to allow right atrium RA and left atrium LA to fully empty of blood prior to the completion of the contraction of heart H. This allows right ventricle RV and left ventricle LV to fill with blood from right atrium RA and left atrium LA, respectively. Atrioventricular node AV then relays the electrical signal through bundle of His HIS and through right bundle branch RBB and left bundle branch LBB. The electrical signal then travels through right bundle branch RBB and left bundle branch LBB to Purkinje fibers PF, which spread the electrical signal through right ventricle RV and left ventricle LV and causes right ventricle RV and left ventricle LV to contract. As right ventricle RV and left ventricle LV contract, blood flows from right ventricle RV into pulmonary artery and from left ventricle LV into aorta AT. The natural electrical system of heart H ensures synchronized contraction of the chambers of heart H, which is essential for efficient blood circulation.

A patient's heartbeat can be recorded using an electrocardiogram (ECG or EKG). The output from the electrocardiogram can be a represented as a waveform. A typical heartbeat includes a P wave that represents atrial depolarization, or contraction of right atrium RA and left atrium LA. The P wave corresponds to the generation of the electrical signal in sinoatrial node SA and the translation of the electrical signal along Bachmann's bundle BB. Following atrial depolarization, a typical heartbeat will include a QRS complex that represents ventricular depolarization, or contraction of left ventricle LV and right ventricle RV. The QRS complex corresponds to the electrical signal transmitting to atrioventricular node AV and then through bundle of His HIS, right bundle branch RBB, left bundle branch LBB, and Purkinje fibers PF.

In a healthy heart, the QRS complex is narrow, as right ventricle RV and left ventricle LV are contracting at the same time. In a healthy heart, the QRS complex is between 60 milliseconds and 90 milliseconds.

When a patient has a traditional pacemaker with a lead implanted in a right ventricular apical (right ventricular pacing), right bundle branch RBB and Purkinje fibers PF extending from right bundle branch RBB are stimulated before left bundle branch LBB and Purkinje fibers PF extending from left bundle branch LBB. The signal received in right bundle branch RBB and Purkinje fibers PF extending from right bundle branch RBB has to travel up bundle of His HIS to atrioventricular node AV and then back down bundle of His HIS to left bundle branch LBB and Purkinje fibers PF extending from left bundle branch LBB. This causes right ventricle RV to contract prior to left ventricle LV, which causes desynchronization between the right side and the left side of heart H. This causes the QRS complex to become widened. The desynchronization is known as pacemaker induced cardiomyopathy, as the desynchronization causes the blood to pump inefficiently in heart H, which can cause enlargement of heart H and lead to heart failure.

More recently, pacemakers have a first lead that is positioned in the right ventricular apical, a second lead positioned in the coronary sinus, and a third lead implanted in the right atrium (biventricular pacing or cardiac resynchronization therapy (CRT)). With biventricular pacing, the pacemaker determines when to pace right ventricle RV and left ventricle LV, causing the pacemaker to time the difference between contraction in right ventricle RV and left ventricle LV. The pacemaker will send an electrical signal (pacing signal) to left ventricle LV first and then right ventricle RV second. The delay between pacing right ventricle RV and left ventricle LV is called an interventricular delay and it is a programmable parameter set by a physician. The challenge with biventricular pacing is that it requires multiple leads, requiring a more complicated delivery procedure and requiring proper imaging. Further, it drains the battery of the pacemaker more quickly, as two chambers of heart H are being paced with every heartbeat.

Additionally, pacemakers can also have a lead that is screwed into interventricular septum IS to pace bundle of His HIS and left bundle branch LBB (left bundle branch pacing). Pacing bundle of His HIS and left bundle branch LBB can better synchronize the contraction of right ventricle RV and left ventricle LV compared to traditional right ventricular pacing. However, the procedure to implant the pacemaker leads is a challenging procedure, requiring physician skill and proper imaging. Further, puncturing interventricular septum IS with a lead creates a risk of injury to heart H.

### Subcutaneous Device 2200

FIG. 2A is a side view of subcutaneous device 2200 anchored to structural body component A. FIG. 2B is a top perspective view of subcutaneous device 2200. FIG. 2C is a side view of subcutaneous device 2200. FIG. 2D is a perspective side view of subcutaneous device 2200 positioned on xiphoid process X and/or sternum S and showing a positioning of prong 2206 on heart H. For ease and clarity of discussion, FIG. 2A-2D will be described together. Subcutaneous device 2200 includes housing 2202, clip 2204, and prong 2206. Housing 2202 includes first side 2210, second side 2212, top side 2214, bottom side 2216, front end 2218, back end 2220, housing latch 2222, and guide 2230. Clip 2204 includes top portion 2240, bottom portion 2242, and tines 2244. Prong 2206 includes proximal end 2260, distal end 2262, base portion 2264, arm portion 2268, contact portion 2270, electrode 2272, sleeve 2274 (which includes upper portion 2276 and lower portion 2278), wire 2280, structural tube 2282, and structural tube 2284. FIG. 2A also shows structural body component A and remote body component B. FIG. 2D further shows xiphoid process X, sternum S, and heart H.

Subcutaneous device 2200 is a medical device that is configured to be anchored to structural body component A (as shown in FIG. 2A), which may be a muscle, a bone, or a tissue of a patient. For example, as shown in FIG. 2D, subcutaneous device 2200 can be anchored to xiphoid process X and/or sternum S. Subcutaneous device 2200 can be a monitoring device, a diagnostic device, and a therapeutic device. For example, subcutaneous device 2200 can be a pacemaker device that is capable of monitoring a patient's heart rate, diagnosing an arrhythmia of the patient's heart, and providing therapeutic electrical stimulation to the patient's heart. Subcutaneous device 2200 includes housing 2202. Housing 2202 of subcutaneous device 2200 may include sensing circuitry, a controller, a memory, a therapy circuitry, electrode(s), sensor(s), a transceiver, and a power source.

Clip 2204 is attached to housing 2202. Clip 2204 is configured to anchor subcutaneous device 2200 to structural body component A. Clip 2204 moves vertically within housing 2202 between an open position and a closed position. Clip 2204 is moved vertically away from housing 2202 when clip 2204 is in an open position. Clip 2204 will be in an open position as it is advanced around structural body component A. Clip 2204 is an active clip. In addition to using the stiffness of clamping components to attach to the bone, the muscle, or the tissue, clip 2204 uses an active fixation method such as tines and/or screws, and/or any other suitable anchoring structure to secure clip 2204 to the bone, the muscle, or the tissue. Clip 2204 is moved vertically toward housing 2202 to change clip 2204 from an open position to a closed position. Clip 2204 is shown in FIG. 2A in a closed position around structural body component A to clamp around structural body component A and anchor subcutaneous device 2200 to structural body component A.

Prong 2206 is connected to and extends away from housing 2202 of subcutaneous device 2200. Prong 2206 is configured to contact remote body component B that is positioned away from structural body component A. Remote body component B may be an organ, a nerve, or tissue of the patient. For example, remote body component B can be the heart of a patient. Prong 2206 includes an electrode that is capable of sensing an electrical activity or physiological parameter of remote body component B and/or providing therapeutic electrical stimulation to remote body component B.

In one example, subcutaneous device 2200 can be a pacemaker and the one or more electrodes on prong 2206 of subcutaneous device 2200 can sense the electrical activity of a heart. The sensed electrical activity can be transmitted to sensing circuitry and a controller in housing 2202 of subcutaneous device 2200. The controller can determine the heart rate of the patient and can detect whether an arrhythmia is present. If an arrhythmia is detected, the controller can send instructions to therapeutic circuitry to provide a therapeutic electrical stimulation to the heart. In this manner, subcutaneous device 2200 functions as a monitoring device, a diagnostic device, and a therapeutic device.

Subcutaneous device 2200 will be discussed as a pacemaker that can be used for monitoring, diagnostics, and therapeutics. In this embodiment, subcutaneous device 2200 is a unipolar pacemaker. In alternate embodiments, subcutaneous device 2200 may be a bipolar pacemaker. Subcutaneous device 2200 can also be an implantable cardioverter-defibrillator.

Housing 2202 can be made out of stainless steel, titanium, nitinol, epoxy, silicone, polyurethane with metallic reinforcements, or any other material that is suitable for non-porous implants. Housing 2202 can also include an exterior coating. Clip 2204 can be made out of stainless steel, titanium, nitinol, epoxy, silicone, polyurethane with metallic reinforcements, or any other material that is suitable for non-porous implants. Prong 2206 can be made out of nickel titanium, also known as Nitinol. Nitinol is a shape memory alloy with superelasticity, allowing prong 2206 to go back to its original shape and position if prong 2206 is deformed as subcutaneous device 2200 is implanted into a patient. Prong 2206 can also be made out of silicone, polyurethane, stainless steel, titanium, epoxy, polyurethane with metallic reinforcements, or any other material that is suitable for non-porous implants. As an example, prong 2206 can be made out of a composite made of polyurethane and silicone and reinforced with metal to provide spring stiffness.

Housing 2202 includes first side 2210, second side 2212, top side 2214, bottom side 2216, front end 2218, back end 2220, housing latch 2222, and guide 2230. First side 2210 is opposite of second side 2212. Top side 2214 is a top of housing 2202 opposite of bottom side 2216, which is a bottom of housing 2202. Front end 2218 is opposite of back end 2220. Housing 2202 is substantially rectangular-shaped in the embodiment shown. In alternate embodiments, housing 2202 can be shaped as a cone, frustum, or cylinder. Housing 2202 can be made out of stainless steel, titanium, Nitinol, epoxy, silicone, polyurethane with metallic reinforcements, or any other material that is suitable for non-porous implants. Housing 2202 can also include an exterior coating.

Housing latch 2222 is connected to back end 2220 of housing 2202. Housing latch 2222 has a top portion that extends along back end 2220 of housing and a bottom portion that extends along bottom side 2216 of housing 2220. The top portion of housing latch 2222 is configured to engage with clip 2204. The bottom portion of housing latch 2222 is curved to accept prong 2206. As such, housing latch 2222 engages with clip 2204 along back end 2220 of housing and with prong 2206 along bottom side 2216 of housing 2202. Housing latch 2222 is configured to attach prong 2206 to bottom side 2216 of housing 2202. Guide 2230 is an L-shaped rod that is connected to back end 2220 and first side 2210 of housing 2202. In this embodiment, guide 2230 is closer to top side 2214 than bottom side 2216 of housing 2202. Guide 2230 is configured to guide housing 2202 of subcutaneous device 2200 through a surgical instrument used to implant subcutaneous device 2200 into a patient.

Clip 2204 includes top portion 2240, bottom portion 2242, and tines 2244. Top portion 2240 is connected to bottom portion 2242. Top portion 2240 forms a top of clip 2204 and is a flat portion of clip 2204 that extends across top side 2214 of housing 2202. Bottom portion 2242 forms a bottom of clip 2204 and is a flat portion that extends along back end 2220 of housing 2202. Bottom portion 2242 of clip 2204 is configured to be attached to housing 2202 and mate with housing latch 2222. Bottom portion 2242 of clip 2204 has pins extending from a back end that are configured to engage with slots in the top portion of housing latch 2222. As such, clip 2204 is connected to housing 2202 via housing latch 2222. Tines 2244 extend from top portion 2240 of clip 2204. Tines 2244 have first ends connected to a center portion of top portion 2240 and second ends that extend away from top portion 2240 toward top side 2214 of housing 2202. Tines 2244 are curved and extend in different directions. Tines 2244 are thin and may be made of metal or any other suitable material. In this embodiment, clip 2204 has four tines 2244. In alternate embodiments, clip 2204 may have any number of tines 2244. Further, in alternate embodiments, any other suitable anchoring structures or active fixation methods may be used along with or instead of tines 2244. Tines 2244 are configured to pierce and anchor to structural body component A.

When clip 2204 is connected to back end 2220 of housing 2202, top portion 2240 of clip 2204 extends along top side 2214 of housing 2202. In this embodiment, top portion 2240 of clip 2204 extends at an angle to the length of housing 2202 from back end 2220 to front end 2218. In alternate embodiments, top portion 2240 of clip 2204 may extend at any angle to the length of housing 2202.

An opening is formed between top portion 2240 of clip 2204 and top side 2214 of housing 2202. Clip 2204 is movable between an open position and a closed position to change the height of the opening. When clip 2204 is in an open position, the opening is expanded and subcutaneous device 2200 is inserted into a patient such that the opening is positioned around the muscle, the bone, or the tissue. After subcutaneous device 2200 is positioned on the muscle, the bone, or the tissue, clip 2204 is moved into a closed position. When clip 2204 is in a closed position, the opening is reduced. Bottom portion 2242 of clip 2204 and housing latch 2222 form a ratchet mechanism to move clip 2204 into the open and closed positions. Top portion 2240 of clip 2204 is forced toward top side 2214 of housing 2202 and down onto the muscle, the bone, or the tissue. Tines 2244 attach to the muscle, the bone, or the tissue, which anchors clip 2204 to the muscle, the bone, or the tissue. Tines 2244 will pierce the muscle, the bone, or the tissue in response to pressure from the engagement of bottom portion 2242 of clip 2204 with housing latch 2222. Tines 2244 may contact top side 2214 of housing such that tines 2244 bend back around into the muscle, the bone, or the tissue and further secure and anchor clip 2204 and subcutaneous device 2200 to the muscle, the bone, or the tissue. Tines 2244 are also removable from the muscle, the bone, or the tissue, such that subcutaneous device 2200 is easily removable from structural body component A.

Prong 2206 includes proximal end 2260 and distal end 2262, which is opposite of proximal end 2260. Prong 2206 includes base portion 2264, arm portion 2268, and contact portion 2270. A first end of base portion 2264 is aligned with proximal end 2260 of prong 2206, and a second end of base portion 2264 is connected to a first end of arm portion 2268. Base portion 2264 is a straight, planar portion that is positioned against and extends along bottom side 2216 of housing 2202. Base portion 2264 is attached to housing 2202. Housing latch 2222 extends around base portion 2264 of prong 2206 to secure base portion 2264 of prong 2206 to housing 2202. Base portion 2264 extends through housing latch 2222. As such, proximal end 2260 of prong 2206 is attached to housing 2202. Base portion 2264 of prong 2206 is electrically connected to the internal components of housing 2202, for example with a feedthrough, to which prong 2206 is also connected.

The first end of arm portion 2268 is connected to the second end of base portion 2264, and a second end of arm portion 2268 is connected to a first end of contact portion 2270. As such, arm portion 2268 extends from base portion 2264 so as to define a first plane that includes opposite ends, or first end and second end, of arm portion 2268 and is perpendicular to the horizontal plane of housing 2202 such that the first plane is a vertical plane that bisects housing 2202 longitudinally from front end 2218 to back end 2220 and is perpendicular to top side 2214 and bottom side 2216. Arm portion 2268 also extends past front end 2218 of housing 2202 so that contact portion 2270 is positioned outwards from front end 2218 of housing 2202. In this embodiment, arm portion 2268 is a predominantly straight portion that is planar and aligned with base portion 2264. The first end of arm portion 2268 acts as a spring for prong 2206 and is under tension. Arm portion 2268 acts as a tension arm and the forces from the first end of arm portion 2268 translate to and push down on the second end of arm portion 2268. As such, prong 2206 undergoes spring action in a vertical plane perpendicular to the horizontal plane of housing 2202 and has a reduced spring action in a horizontal plane parallel to the horizontal plane of housing 2202 due to high lateral stiffness of the planar arm portion 2268. In alternate embodiments, arm portion 2268 of prong 2206 can extend from housing 2202 in any direction or directions.

The first end of contact portion 2270 is connected to the second end of arm portion 2268, and a second end of contact portion 2270 is aligned with distal end 2262 of prong 2206. As such, arm portion 2268 is between base portion 2264 and contact portion 2270. Arm portion 2268 extends beyond front end 2218 of housing 2202 so that contact portion 2270 is positioned beyond front end 2218 of housing 2202. Contact portion 2270 can be positioned such that distal end 2262 of prong 2206 contacts remote body component B. Contact portion 2270 is angled with respect to housing 2202 and arm portion 2268. Contact portion 2270 is angled away from the first plane defined with respect to the arm portion 2268 and housing 2202. In this embodiment, contact portion 2270 is angled away from bottom side 2216 of housing 2202. Contact portion 2270 is also curved, or angled, away from first side 2210 of housing 2202. Contact portion 2270 extends away from bottom side 2216 of housing 2202 and extends away from first side 2210 of housing 2202 so that distal end 2262 of prong 2206 is positioned below and away from housing 2202 and arm portion 2268. In alternate embodiments, contact portion 2270 may be angled in any direction with respect to bottom side 2216 of housing and in any direction with respect to first side 2210 and second side 2212 of housing 2202 depending on the location of remote body component B with respect to structural body component A. Contact portion 2270 is angled toward remote body component B. For example, when remote body component B is a lung or a kidney, contact portion 2270 is angled toward the lung or the kidney. In this embodiment, a first portion of contact portion 2270 is angled about 90 degrees from bottom side 2216 of housing 2202 and arm portion 2268, and a second portion of contact portion 2270 is angled about 90 degrees from first side 2210 of housing 2202 and arm portion 2268. Contact portion 2270 may be angled about 45 degrees to about 60 degrees from the first vertical plane defined with respect to arm portion 2268 and housing 2202.

Prong 2206 further includes electrode 2272. Electrode 2272 is at distal end 2262 of prong 2206. As such, electrode 2272 makes up the second end of contact portion 2270. Electrode 2272 has a cylindrical portion on a first end and a spherical portion on a second end. The cylindrical portion is connected to the spherical portion. The cylindrical portion of the electrode is positioned within the second end of wire 2280. The spherical portion is positioned outside of wire 2280. The spherical portion defines distal end 2260 of prong 2206. The cylindrical portion connects electrode 2272 to wire 2280. The spherical portion positions electrode 2272 at the end of wire 2280, acting as a stop for electrode 2272. The spherical portion is configured to contact remote body component B. For example, spherical portion 1688A buries into the surface of the heart when subcutaneous device 2200 is positioned on the xiphoid process X and/or sternum S of a patient, as shown in FIG. 2D. The spherical portion is rounded such that electrode 2272 is rounded and not sharp where electrode 2272 contacts remote body component B. For example, when electrode 2272 presses against the heart, electrode 2272 is not stiff or sharp enough to piece or tear the pericardial or epicardial tissue.

Electrode 2272 is conductive without significantly decreasing impedance and is shaped to allow for optimal contact with remote body component B without piercing remote body component B. The rounded spherical portion of electrode 2272 prevents electrode 2272 from puncturing or damaging the heart. As such, prong 2206 can have sufficient stiffness and apply enough pressure to remote body component B to maintain constant contact between electrode 2272 and remote body component B without causing damage to remote body component B. For example, when remote body component B is the heart, electrode 2272 does not puncture the heart and cause damage to the heart as the heart beats.

In alternate embodiments, electrode 2272 may have any other suitable shape. Prong 2206 has a single electrode 2272 in the embodiment shown in FIGS. 2A-2D. Prong 2206 can have any number of electrodes in alternate embodiments. Electrode 2272 is positioned at distal end 2262 of prong 2206 to sense an electrical activity or physiological status of remote body component B. Electrode 2272 can also provide therapeutic electrical stimulation to remote body component B.

Sleeve 2274 is a hollow outer portion of prong 2206. Sleeve 2274 extends from proximal end 2260 of prong 2260 to contact portion 2270. A first end of sleeve 2274 is aligned with proximal end 2260 of prong 2206. Sleeve 2274 extends along base portion 2264, arm portion 2268, and a first portion of contact portion 2270. A second end of sleeve 2274 is within contact portion 2270. As such, sleeve 2274 makes up the outer portions of base portion 2264, arm portion 2268, and the first portion of contact portion 2270. Sleeve 2274 has upper portion 2276 opposite lower portion 2278. Upper portion 2276 and lower portion 2278 are flat, or planar, such that sleeve 2274 has a flat, or generally rectangular, cross-section. As such, a majority of prong 2206 has a flat, or generally rectangular, cross-section.

Wire 2280 extends through sleeve 2274, between upper portion 2276 and lower portion 2278, from proximal end 2260 of prong 2206 to contact portion 2270. Wire 2280 extends beyond the second end of sleeve 2274. A first end of wire 2280 is aligned with proximal end 2260 of prong 2206. Wire 2280 extends along base portion 2264, arm portion 2268, and contact portion 2270. A second end of wire 2280 is connected to electrode 2272. As such, contact portion 2270 of prong 2206 is made up of sleeve 2274, wire 2280, and electrode 2272. Wire 2280 has the same overall shape and angle as sleeve 2274 and extends beyond the second end of sleeve 2274. Wire 2280 extends away from the second end of sleeve 2274 and first side 2210 of housing 2202. In this embodiment, wire 2280 extends about 90 degrees away from the second end of sleeve 2274. As such, in this embodiment, wire 2280 is angled away from bottom side 2216 of housing 2202 along with sleeve 2274 and curved, or angled, away from first side 2210 of housing 2202 beyond sleeve 2274.

Structural tubes 2282 and 2284 extend through sleeve 2274, between upper portion 2276 and lower portion 2278 and along wire 2280. Structural tubes 2282 and 2284 extend from proximal end 2260 of prong 2260 to the second end of arm portion 2268. First ends of structural tubes 2282 and 2284 are aligned with proximal end 2260 of prong 2206. Structural tubes 2282 and 2284 extend along base portion 2264 and arm portion 2268. Second ends of structural tubes 2282 and 2284 are aligned with the second end of arm portion 2268. In alternate embodiments, structural tubes 2282 and 2284 may extend into contact portion 2270 to the second end of sleeve 2274 such that second ends of structural tubes 2282 and 2284 are aligned with the second end of sleeve 2274. Structural tubes 2282 and 2284 have the same overall shape as base portion 2264 and arm portion 2268. As such, in this embodiment, structural tubes 2282 and 2284 are planar.

First structural tube 2282 is on a first side of wire 2280, and second structural tube 2284 is on a second side of wire 2280 such that wire 2280 has structural tubes 2282 and 2284 on opposite sides of wire 2280. In alternate embodiments, prong 2206 may include any number of structural tubes 2282 and 2284 based on the desired stiffness of prong 2206. Structural tubes 2282 and 2284 may be hollow or solid. Structural tubes 2282 and 2284 can be any suitable size. For example, structural tubes 2282 and 2284 can have the same diameters as each other, can have the same diameter as wire 2280, or can have a smaller diameter than wire 2280. Structural tubes 2282 and 2284 can have any suitable thickness based on desired stiffness of prong 2206. Structural tubes 2282 and 2284 may be made of metal, polyurethane, silicone, any suitable plastic, a combination of metal and plastic, or any other suitable material. Structural tubes 2282 and 2284 are limited to an amount of metal that allows subcutaneous device 2200 to be MRI compatible. In alternate embodiments, prong 2206 may include any number of structural tubes 2282 and 2284. The size, shape, and material of structural tubes 2282 and 2284 may be selected based upon the desired stiffness of prong 2206. For example, prong 2206 may include five, seven, or any other suitable number of structural tubes 2282 and 2284 to make prong 2206 flatter and increase the stiffness of prong 2206.

Prong 2206 is angled with respect to housing 2202 to improve contact of electrode 2272 with remote body component B. Prong 2206 is angled so that contact portion 2270 pushes down against remote body component B, such as the heart. Electrode 2272 at distal end 2262 of prong 2206 contacts the heart and buries into the cardiac tissue. Further, because prong 2206 is angled down toward heart, prong 2206 applies pressure to the heart as the heart beats and moves up and down, without increasing the stiffness of prong 2206. As a result, electrode 2272 maintains contact with the heart without fixing electrode 2272 to the heart. For example, prong 2206 is prevented from bouncing off of the heart as the heart beats, which would cause intermittent contact that reduces functionality. Additionally, contact portion 2270 is angled away from bottom 2216 and first side 2210 of housing 2202 to ensure distal end 2262 of prong 2206 is positioned on the heart when subcutaneous device 2200 is attached to xiphoid process X and/or sternum S of a patient, as shown in FIG. 2D. As such, subcutaneous device 2200 can be inserted and deployed into a patient without requiring cardiac catheterization labs. Thus, the procedure for inserting the device is simple and only requires local anesthesia, which means it can be carried out in various environments, such as in an ambulance.

Arm portion 2268 of prong 2206 allows prong 2206 to be flexible once it is positioned in the body. The pivot point of arm portion 2268 is adjacent the first end of arm portion 2268, which is connected to the second end of base portion 2264, and slightly closer to proximal end 2260 than front end 2218 of housing 2202 is to proximal end 2206, or where prong 2206 is secured to bottom side 2216 of housing 2202 by housing latch 2222. For example, if remote body component B is the heart of the patient and contact portion 2270 of prong 2206 is positioned against the heart, arm portion 2268 of prong 2206 allows prong 2206 to move up and down with the heart as the heart beats. This ensures that prong 2206 does not puncture or damage the heart while contact portion 2270 of prong 2206 maintains contact with the heart. In this embodiment, electrode 2272 at distal end 2262 of prong 2206 has a rounded end to further prevent prong 2206 from puncturing or damaging the heart when contact portion 2270 of prong 2206 is in contact with the heart. The overall axial stiffness of prong 2206 can be adjusted so that prong 2206 gently presses against the heart and moves up and down in contact with the heart as the heart beats, but is not stiff or sharp enough to pierce or tear the pericardial or epicardial tissue. For example, the overall axial stiffness of prong 2206 can be adjusted by adjusting the material of prong 2206, the spring bias or mechanical resistance of prong 2206, the cross-sectional thickness of prong 2206, the angle of incidence of prong 2206 on remote body component B, the outer profile of prong 2206 where prong 2206 contacts remote body component B, and/or any other suitable characteristic of prong 2206.

The flat, or rectangular, cross-section of sleeve 2274 created by planar upper portion 2276 and planar lower portion 2278 provides stiffness to prong 2206, which makes prong 2206 more resistant to in-plane bending. Sleeve 2274 also provides space for wire 2280 to be surrounded by structural tubes 2282 and 2284. Structural tubes 2282 and 2284 also provide the desired structural stiffness to prong 2206. As a result, prong 2206 resists in-plane bending, or bending in any direction, to maintain positioning with respect to the heart, which ensures electrode 2272 maintains contact with the heart without requiring fluoroscopy or other visualization tools. In alternate embodiments, prong 2206 may include a pre-shaped spine made of shape-memory material, such as nitinol, to provide stiffness along with or instead of structural tubes 2282 and 2284. In these embodiments, prong 2206 may have the shape shown in FIG. 2A-2D, for example, or other suitable shapes or configurations.

Subcutaneous device 2200 is described here as having a single prong 2206. In alternate embodiments, subcutaneous device 2200 can include any number of prongs and those prongs can have any shape. Contact portion 2270 can have any angle with respect to bottom side 2216, first side 2210, and second side 2212 of housing 2202.

As shown in FIG. 2D, subcutaneous device 2200 can be anchored to xiphoid process X and sternum S of a patient. Subcutaneous device 2200 can be implanted with a simple procedure where subcutaneous device 2200 is injected onto xiphoid process X and sternum S using a surgical instrument. Subcutaneous device 2200 can function as a pacemaker. Prong 2206 can be shaped so that contact portion 2270 of prong 2206 contacts the Manicka Zone of Pacing of heart H. Subcutaneous device 2200 can function as a unipolar pacemaker, utilizing electrode 2272 on prong 2206. Further, subcutaneous device 2200 can function as a bipolar pacemaker, utilizing more than one prong 2206 and/or electrode 2272.

### Subcutaneous Device 2300

FIG. 3A is a side view of subcutaneous device 2300 anchored to structural body component A. FIG. 3B is a perspective view of subcutaneous device 2300. FIG. 3C is a side view of subcutaneous device 2300. FIG. 3D is a perspective view of subcutaneous device 2300 positioned on xiphoid process X and/or sternum S and showing a positioning of prongs 2306 and 2306A on heart H. For ease and clarity of discussion, FIG. 3A-3D will be described together. Subcutaneous device 2300 includes housing 2302, clip 2304, prong 2306, and prong 2306A. Housing 2302 includes first side 2310, second side 2312, top side 2314, bottom side 2316, front 109 end 2318, back end 2320, housing latch 2322, and guide 2330. Clip 2304 includes top portion 2340, bottom portion 2342, and tines 2344. Prong 2306 includes proximal end 2360, distal end 2362, base portion 2364, arm portion 2368, contact portion 2370, electrode 2372, sleeve 2374 (which includes upper portion 2376 and lower portion 2378), wire 2380, structural tube 2382, and structural tube 2384. Prong 2306A includes proximal end 2360A, distal end 2362A, base portion 2364A, arm portion 2368A, contact portion 2370A, electrode 2372A, sleeve 2374A (which includes upper portion 2376A and lower portion 2378A), wire 2380A, structural tube 2382A, and structural tube 2384A. FIG. 3A also shows structural body component A and remote body component B. FIG. 3D further shows xiphoid process X, sternum S, and heart H.

Subcutaneous device 2300 is a medical device that is configured to be anchored to structural body component A (as shown in FIG. 3A), which may be a muscle, a bone, or a tissue of a patient. For example, as shown in FIG. 3D, subcutaneous device 2300 can be anchored to xiphoid process X and/or sternum S. Subcutaneous device 2300 can be a monitoring device, a diagnostic device, and a therapeutic device. For example, subcutaneous device 2300 can be a pacemaker device that is capable of monitoring a patient's heart rate, diagnosing an arrhythmia of the patient's heart, and providing therapeutic electrical stimulation to the patient's heart. Subcutaneous device 2300 includes housing 2302. Housing 2302 of subcutaneous device 2300 may include sensing circuitry, controller, memory, therapy circuitry, electrode(s), sensor(s), transceiver, and power source and/or any other component of a medical device.

Clip 2304 is attached to housing 2302. Clip 2304 is configured to anchor subcutaneous device 2300 to structural body component A. Clip 2304 moves vertically within housing 2302 between an open position and a closed position. Clip 2304 is moved vertically away from housing 2302 when clip 2304 is in an open position. Clip 2304 will be in an open position as it is advanced around structural body component A. Clip 2304 is an active clip. In addition to using the stiffness of clamping components to attach to the bone, the muscle, or the tissue, clip 2304 uses an active fixation method such as tines and/or screws, and/or any other suitable anchoring structure to secure clip 2304 to the bone, the muscle, or the tissue. Clip 2304 is moved vertically toward housing 2302 to change clip 2304 from an open position to a closed position. Clip 2304 is shown in a closed position around structural body component A to clamp around structural body component A and anchor subcutaneous device 2300 to structural body component A.

Prong 2306 and prong 2306A are connected to and extend away from housing 2302 of subcutaneous device 2300. In the embodiment shown in FIGS. 3A-3D, prong 2306A is positioned above prong 2306 such that prong 2306A is between prong 2306 and housing 2302. In alternate embodiments, prong 2306A may be positioned next to prong 2306. Prong 2306 and prong 2306A are configured to contact remote body component B that is positioned away from structural body component A. In alternate embodiments, prong 2306A may be configured to contact a different remote body component B that is positioned away from structural body component A and remote body component B that prong 2306 contacts. Remote body component B may be an organ, a nerve, or tissue of the patient. For example, remote body component B can be the heart of the patient. Prong 2306 and prong 2306A each include an electrode that is capable of sensing an electrical activity or physiological parameter of remote body component B and/or providing therapeutic electrical stimulation to remote body component B.

In one example, subcutaneous device 2300 can be a pacemaker and the electrodes on prong 2306 and prong 2306A of subcutaneous device 2300 can sense the electrical activity of a heart. The sensed electrical activity can be transmitted to sensing circuitry and a controller in housing 2302 of subcutaneous device 2300. The controller can determine the heart rate of the patient and can detect whether an arrhythmia is present. If an arrhythmia is detected, the controller can send instructions to therapeutic circuitry to provide a therapeutic electrical stimulation to the heart. In this manner, subcutaneous device 2300 functions as a monitoring device, a diagnostic device, and a therapeutic device.

Subcutaneous device 2300 will be discussed as a pacemaker that can be used for monitoring, diagnostics, and therapeutics. In this embodiment, subcutaneous device 2300 is a unipolar pacemaker. In alternate embodiments, subcutaneous device 2300 may be a bipolar pacemaker. Subcutaneous device 2300 can also be an implantable cardioverter-defibrillator.

Housing 2302 can be made out of stainless steel, titanium, nitinol, epoxy, silicone, polyurethane with metallic reinforcements, or any other material that is suitable for non-porous implants. Housing 2302 can also include an exterior coating. Clip 2304 can be made out of stainless steel, titanium, nitinol, epoxy, silicone, polyurethane with metallic reinforcements, or any other material that is suitable for non-porous implants. Prongs 2306 and 2306A can be made out of nickel titanium, also known as Nitinol. Nitinol is a shape memory alloy with superelasticity, allowing prongs 2306 and 2306A to go back to their original shapes and positions if prongs 2306 and/or 2306A are deformed as subcutaneous device 2300 is implanted into a patient. Prong 2306 and 2306A can also be made out of silicone, polyurethane, stainless steel, titanium, epoxy, polyurethane with metallic reinforcements, or any other material that is suitable for non-porous implants. As an example, prong 2306 and/or prong 2306A can be made out of a composite made of polyurethane and silicone and reinforced with metal to provide spring stiffness.

Housing 2302 includes first side 2310, second side 2312, top side 2314, bottom side 2316, front end 2318, back end 2320, housing latch 2322, and guide 2330. First side 2310 is opposite of second side 2312. Top side 2314 is a top of housing 2302 opposite of bottom side 2316, which is a bottom of housing 2302. Bottom side 2316 of housing 2302 may be shaped to form a channel that accepts prong 2306A, or prong 2306A may be attached to bottom side 2316 of housing 2302 with a latch. Prongs 2306 and 2306A may also be positioned side-by-side and attached to bottom side 2316 of housing 2302 with a latch, or within a channel. Front end 2318 is opposite of back end 2320. Housing 2302 is substantially rectangular-shaped in the embodiment shown. In alternate embodiments, housing 2302 can be shaped as a cone, frustum, or cylinder. Housing 2302 can be made out of 110 stainless steel, titanium, Nitinol, epoxy, silicone, polyurethane with metallic reinforcements, or any other material that is suitable for non-porous implants. Housing 2302 can also include an exterior coating.

Housing latch 2322 is connected to back end 2320 of housing 2302. Housing latch 2322 has a top portion that extends along back end 2320 of housing and a bottom portion that extends along bottom side 2316 of housing 2320. The top portion of housing latch 2322 is configured to engage with clip 2304. The bottom portion of housing latch 2322 is curved to accept prong 2306, and in some embodiments prong 2306A. For example, housing latch 2322 may accept prong 2306 and prong 2306A when prong 2306 and prong 2306A are positioned side-by-side. As such, housing latch 2322 engages with clip 2304 along back end 2320 of housing and with prong 2306, and sometimes prong 2306A, along bottom side 2316 of housing 2302. Housing latch 2322 is configured to attach prongs 2306 and 2306A to bottom side 2216 of housing 2302. Guide 2330 is an L-shaped rod that is connected to back end 2320 and first side 2310 of housing 2302. In this embodiment, guide 2330 is closer to top side 2314 than bottom side 2316 of housing 2302. Guide 2330 is configured to guide housing 2302 of subcutaneous device 2300 through a surgical instrument used to implant subcutaneous device 2300 into a patient.

Clip 2304 includes top portion 2340, bottom portion 2342, and tines 2344. Top portion 2340 is connected to bottom portion 2342. Top portion 2340 forms a top of clip 2304 and is a flat portion of clip 2304 that extends across top side 2314 of housing 2302. Bottom portion 2342 forms a bottom of clip 2304 and is a flat portion that extends along back end 2320 of housing 2302. Bottom portion 2342 of clip 2304 is configured to be attached to housing 2302 and mate with housing latch 2322. Bottom portion 2342 of clip 2304 has pins extending from a back end that are configured to engage with slots in the top portion of housing latch 2322. As such, clip 2304 is connected to housing 2302 via housing latch 2322. Tines 2344 extend from top portion 2340 of clip 2304. Tines 2344 have first ends connected to a center portion of top portion 2340 and second ends that extend away from top portion 2340 toward top side 2314 of housing 2302. Tines 2344 are curved and extend in different directions. Tines 2344 are thin and may be made of metal or any other suitable material. In this embodiment, clip 2304 has four tines 2344. In alternate embodiments, clip 2304 may have any number of tines 2344. Further, in alternate embodiments, any other suitable anchoring structures or active fixation methods may be 111 used along with or instead of tines 2344. Tines 2344 are configured to pierce and anchor to structural body component A.

When clip 2304 is connected to back end 2320 of housing 2302, top portion 2340 of clip 2304 extends along top side 2314 of housing 2302. In this embodiment, top portion 2340 of clip 2304 extends at an angle to the length of housing 2302 from back end 2320 to front end 2318. In alternate embodiments, top portion 2340 of clip 2304 may extend at any angle to the length of housing 2302.

An opening is formed between top portion 2340 of clip 2304 and top side 2314 of housing 2302. Clip 2304 is movable between an open position and a closed position to change the height of the opening. When clip 2304 is in an open position, the opening is expanded and subcutaneous device 2300 is inserted into a patient such that the opening is positioned around the muscle, the bone, or the tissue. After subcutaneous device 2300 is positioned on the muscle, the bone, or the tissue, clip 2304 is moved into a closed position. When clip 2304 is in a closed position, the opening is reduced. Bottom portion 2342 of clip 2304 and housing latch 2322 form a ratchet mechanism to move clip 2304 into the open and closed positions. Top portion 2340 of clip 2304 is forced toward top side 2314 of housing 2302 and down onto the muscle, the bone, or the tissue. Tines 2344 attach to the muscle, the bone, or the tissue, which anchors clip 2304 to the muscle, the bone, or the tissue. Tines 2344 will pierce the muscle, the bone, or the tissue in response to pressure from the engagement of bottom portion 2342 of clip 2304 with housing latch 2322. Tines 2344 may contact top side 2314 of housing such that tines 2344 bend back around into the muscle, the bone, or the tissue and further secure and anchor clip 2304 and subcutaneous device 2300 to the muscle, the bone, or the tissue. Tines 2344 are also removable from the muscle, the bone, or the tissue, such that subcutaneous device 2300 is easily removable from structural body component A.

Prong 2306 includes proximal end 2360 and distal end 2362, which is opposite of proximal end 2360. Prong 2306 includes base portion 2364, arm portion 2368, and contact portion 2370. A first end of base portion 2364 is aligned with proximal end 2360 of prong 2306, and a second end of base portion 2364 is connected to a first end of arm portion 2368. Base portion 2364 is a straight, planar portion that is positioned against and extends along bottom side 2316 of housing 2302 and a bottom of prong 2306A when prong 2306A is positioned above prong 2306. Base portion 2364 is attached to housing 2302. Housing latch 2322 extends around base portion 2364 of prong 2306 to secure base portion 2364 of prong 2306 to housing 2302. 112 Base portion 2364 extends through housing latch 2322. As such, proximal end 2360 of prong 2306 is attached to housing 2302. Base portion 2364 of prong 2306 is electrically connected to the internal components of housing 2302, for example with a feedthrough, to which prong 2306 is also connected.

The first end of arm portion 2368 is connected to the second end of base portion 2364, and a second end of arm portion 2368 is connected to a first end of contact portion 2370. As such, arm portion 2368 extends from base portion 2364 so as to define a first plane that includes opposite ends, or first end and second end, of arm portion 2368 and is perpendicular to the horizontal plane of housing 2302 such that the first plane is a vertical plane that bisects housing 1602 longitudinally from front end 1618 to back end 1620 and is perpendicular to top side 1614 and bottom side 1616. Arm portion 2368 also extends past front end 2318 of housing 2302 so that contact portion 2370 is positioned outwards from front end 2318 of housing 2302. In this embodiment, arm portion 2368 is a predominantly straight portion that is planar and aligned with base portion 2364. The first end of arm portion 2368 acts as a spring for prong 2306 and is under tension. Arm portion 2368 acts as a tension arm and the forces from the first end of arm portion 2368 translate to and push down on the second end of arm portion 2368. In alternate embodiments, arm portion 2368 of prong 2306 can extend from housing 2302 in any direction or directions.

The first end of contact portion 2370 is connected to the second end of arm portion 2368, and a second end of contact portion 2370 is aligned with distal end 2362 of prong 2306. As such, arm portion 2368 is between base portion 2364 and contact portion 2370. Arm portion 2368 extends beyond front end 2318 of housing 2302 so that contact portion 2370 is positioned beyond front end 2318 of housing 2302. Contact portion 2370 can be positioned such that distal end 2362 of prong 2306 contacts remote body component B (shown in FIG. 3A). Contact portion 2370 is angled with respect to housing 2302 and arm portion 2368. Contact portion 2370 is angled away from the first plane defined with respect to arm portion 2368 and housing 2302. In this embodiment, contact portion 2370 is angled away from bottom side 2316 of housing 2302. Contact portion 2370 is also curved, or angled, away from first side 2310 of housing 2302. Contact portion 2370 extends away from bottom side 2316 of housing 2302 and extends away from first side 2310 of housing 2302 so that distal end 2362 of prong 2306 is positioned below and away from housing 2302 and arm portion 2368. In alternate embodiments, contact portion 2370 may be angled in any direction with respect to bottom side 2316 of housing 113 and in any direction with respect to first side 2310 and second side 2312 of housing 2302 depending on the location of remote body component B with respect to structural body component A. Contact portion 2370 is angled toward remote body component B. For example, when remote body component B is a lung or a kidney, contact portion 2370 is angled toward the lung or the kidney. In this embodiment, a first portion of contact portion 2370 is angled about 90 degrees from bottom side 2316 of housing 2302 and arm portion 2368, and a second portion of contact portion 2370 is angled about 90 degrees from first side 2310 of housing 2302 and arm portion 2368. Contact portion 2370 may be angled about 45 degrees to about 60 degrees from the first vertical plane defined with respect to arm portion 2368 and housing 2302.

Prong 2306 further includes electrode 2372. Electrode 2372 is at distal end 2362 of prong 2306. As such, electrode 2372 makes up the second end of contact portion 2370. Electrode 2372 has a rounded end and has the same shape as electrode 2272. In alternate embodiments, electrode 2372 may have any suitable shape. Prong 2306 has a single electrode 2372 in the embodiment shown in FIGS. 3A-3D. Prong 2306 can have any number of electrodes in alternate embodiments. Electrode 2372 is positioned at distal end 2362 of prong 2306 to sense an electrical activity or physiological status of remote body component B. Electrode 2372 can also provide therapeutic electrical stimulation to remote body component B.

Sleeve 2374 is a hollow outer portion of prong 2306. Sleeve 2374 extends from proximal end 2360 of prong 2360 to contact portion 2370. A first end of sleeve 2374 is aligned with proximal end 2360 of prong 2306. Sleeve 2374 extends along base portion 2364, arm portion 2368, and a first portion of contact portion 2370. A second end of sleeve 2374 is within contact portion 2370. As such, sleeve 2374 makes up the outer portions of base portion 2364, arm portion 2368, and the first portion of contact portion 2370. Sleeve 2374 has upper portion 2376 opposite lower portion 2378. Upper portion 2376 and lower portion 2378 are flat, or planar, such that sleeve 2374 has a flat, or generally rectangular, cross-section. As such, a majority of prong 2306 has a flat, or generally rectangular, cross-section.

Wire 2380 extends through sleeve 2374, between upper portion 2376 and lower portion 2378, from proximal end 2360 of prong 2306 to contact portion 2370. Wire 2380 extends beyond the second end of sleeve 2374. A first end of wire 2380 is aligned with proximal end 2360 of prong 2306. Wire 2380 extends along base portion 2364, arm portion 2368, and contact portion 2370. A second end of wire 2380 is connected to electrode 2372. As such, 114 contact portion 2370 of prong 2306 is made up of sleeve 2374, wire 2380, and electrode 2372. Wire 2380 has the same overall shape and angle as sleeve 2374 and extends beyond the second end of sleeve 2374. Wire 2380 extends away from the second end of sleeve 2374 and first side 2310 of housing 2302. In this embodiment, wire 2380 extends about 90 degrees away from the second end of sleeve 2374. As such, in this embodiment, wire 2380 is angled away from bottom side 2316 of housing 2302 along with sleeve 2374 and curved, or angled, away from first side 2310 of housing 2302 beyond sleeve 2374.

Structural tubes 2382 and 2384 extend through sleeve 2374, between upper portion 2376 and lower portion 2378 and along wire 2380. Structural tubes 2382 and 2384 extend from proximal end 2360 of prong 2360 to the second end of arm portion 2368. First ends of structural tubes 2382 and 2384 are aligned with proximal end 2360 of prong 2306. Structural tubes 2382 and 2384 extend along base portion 2364 and arm portion 2368. Second ends of structural tubes 2382 and 2384 are aligned with the second end of arm portion 2368. In alternate embodiments, structural tubes 2382 and 2384 may extend into contact portion 2370 to the second end of sleeve 2374 such that second ends of structural tubes 2382 and 2384 are aligned with the second end of sleeve 2374. Structural tubes 2382 and 2384 have the same overall shape as base portion 2364 and arm portion 2368. As such, in this embodiment, structural tubes 2382 and 2384 are planar.

First structural tube 2382 is on a first side of wire 2380, and second structural tube 2384 is on a second side of wire 2380 such that wire 2380 has structural tubes 2382 and 2384 on opposite sides of wire 2380. In alternate embodiments, prong 2306 may include any number of structural tubes 2382 and 2384 based on the desired stiffness of prong 2306. Structural tubes 2382 and 2384 may be hollow or solid. Structural tubes 2382 and 2384 can be any suitable size. For example, structural tubes 2382 and 2384 can have the same diameters as each other, can have the same diameter as wire 2380, or can have a smaller diameter than wire 2380. Structural tubes 2382 and 2384 can have any suitable thickness based on desired stiffness of prong 2306. Structural tubes 2382 and 2384 may be made of metal, polyurethane, silicone, any suitable plastic, a combination of metal and plastic, or any other suitable material. Structural tubes 2382 and 2384 are limited to an amount of metal that allows subcutaneous device 2300 to be MRI compatible. In alternate embodiments, prong 2306 may include any number of structural tubes 2382 and 2384. The size, shape, and material of structural tubes 2382 and 2384 may be selected 115 based upon the desired stiffness of prong 2306. For example, prong 2306 may include five, seven, or any other suitable number of structural tubes 2382 and 2384 to make prong 2306 flatter and increase the stiffness of prong 2306.

Prong 2306A includes proximal end 2360A and distal end 2362A, which is opposite of proximal end 2360A. Prong 2306A includes base portion 2364A, arm portion 2368A, and contact portion 2370A. A first end of base portion 2364A is aligned with proximal end 2360A of prong 2306A, and a second end of base portion 2364A is connected to a first end of arm portion 2368A. Base portion 2364A is a straight, planar portion that is positioned against and extends along bottom side 2316A of housing 2302A, and base portion 2364 of prong 2306 when prong 2306A is positioned above prong 2306. Base portion 2364A is attached to housing 2302. Housing latch 2322A may extend around base portion 2364A of prong 2306A to secure base portion 2364A of prong 2306A to housing 2302A, such as when prong 2306 and prong 2306A are positioned side-by-side. As such, base portion 2364A extends through housing latch 2322A, and proximal end 2360A of prong 2306A is attached to housing 2302A. Base portion 2364A of prong 2306A is electrically connected to the internal components of housing 2302A, for example with a feedthrough, to which prong 2306A is also connected.

The first end of arm portion 2368A is connected to the second end of base portion 2364A, and a second end of arm portion 2368A is connected to a first end of contact portion 2370A. As such, arm portion 2368A extends from base portion 2364A so as to define a second plane that includes opposite ends, or first end and second end, of arm portion 2368A and is perpendicular to the horizontal plane of housing 2302 such that the second plane is a vertical plane that bisects housing 2302 longitudinally from front end 2318 to back end 2320 and is perpendicular to top side 2314 and bottom side 2316. Arm portion 2368A also extends past front end 2318A of housing 2302A so that contact portion 2370A is positioned outwards from front end 2318A of housing 2302A. Arm portion 2368A extends past arm portion 2368 of prong 2306. In this embodiment, arm portion 2368A is a predominantly straight portion that is planar and aligned with base portion 2364A. The first end of arm portion 2368A acts as a spring for prong 2306A and is under tension. Arm portion 2368A acts as a tension arm and the forces from the first end of arm portion 2368A translate to and push down on the second end of arm portion 2368A. In alternate embodiments, arm portion 2368A of prong 2306A can extend from housing 2302A in any direction or directions.

The first end of contact portion 2370A is connected to the second end of arm portion 2368A, and a second end of contact portion 2370A is aligned with distal end 2362A of prong 2306A. As such, arm portion 2368A is between base portion 2364A and contact portion 2370A. Arm portion 2368A extends beyond front end 2318A of housing 2302A so that contact portion 2370A is positioned beyond front end 2318A of housing 2302A. Further, arm portion 2368A extends beyond arm portion 2368 of prong 2306 so that contact portion 2370A extends beyond contact portion 2370 of prong 2306. Contact portion 2370A can be positioned such that distal end 2362A of prong 2306A contacts remote body component B. Contact portion 2370A is angled with respect to housing 2302A and arm portion 2368A. Contact portion 2370A is angled away from the second plane defined with respect to arm portion 2368A and housing 2302. In this embodiment, contact portion 2370A is angled away from bottom side 2316A of housing 2302A. Contact portion 2370A is also curved, or angled, away from first side 2310A of housing 2302A. Contact portion 2370A extends away from bottom side 2316A of housing 2302A and extends away from first side 2310A of housing 2302A so that distal end 2362A of prong 2306A is positioned below and away from housing 2302 and arm portion 2368A. In alternate embodiments, contact portion 2370A may be angled in any direction with respect to bottom side 2316A of housing and in any direction with respect to first side 2310A and second side 2312A of housing 2302A depending on the location of remote body component B with respect to structural body component A. Contact portion 2370A is angled toward remote body component B. For example, when remote body component B is a lung or a kidney, contact portion 2370A is angled toward the lung or the kidney. In this embodiment, a first portion of contact portion 2370A is angled about 90 degrees from bottom side 2316A of housing 2302A and arm portion 2368A, and a second portion of contact portion 2370A is angled about 90 degrees from first side 2310A of housing 2302A and arm portion 2368A. Contact portion 2370A may be angled about 45 degrees to about 60 degrees from the first vertical plane defined with respect to arm portion 2368A and housing 2302.

Prong 2306A further includes electrode 2372A. Electrode 2372A is at distal end 2362A of prong 2306A. As such, electrode 2372A makes up the second end of contact portion 2370A. Electrode 2372A has a rounded end and has the same shape as electrode 2272 described in FIG. 2A-2D. In alternate embodiments, electrode 2372A may have any suitable shape. Prong 2306A has a single electrode 2372A in the embodiment shown in FIGS. 3A-3D. Prong 2306A can have any number of electrodes in alternate embodiments. Electrode 2372A is positioned at distal end 2362A of prong 2306A to sense an electrical activity or physiological status of remote body component B. Electrode 2372A can also provide therapeutic electrical stimulation to remote body component B.

Sleeve 2374A is a hollow outer portion of prong 2306A. Sleeve 2374A extends from proximal end 2360A of prong 2360A to contact portion 2370A. A first end of sleeve 2374A is aligned with proximal end 2360A of prong 2306A. Sleeve 2374A extends along base portion 2364A, arm portion 2368A, and a first portion of contact portion 2370A. A second end of sleeve 2374A is within contact portion 2370A. As such, sleeve 2374A makes up the outer portions of base portion 2364A, arm portion 2368A, and the first portion of contact portion 2370A. Sleeve 2374A has upper portion 2376A opposite lower portion 2378A. Upper portion 2376A and lower portion 2378A are flat, or planar, such that sleeve 2374A has a flat, or generally rectangular, cross-section. As such, a majority of prong 2306A has a flat, or generally rectangular, cross-section. Lower portion 2378A of sleeve 2374A of prong 2306A is adjacent upper portion 2376 of sleeve 2374 of prong 2306.

Wire 2380A extends through sleeve 2374A, between upper portion 2376A and lower portion 2378A, from proximal end 2360A of prong 2306A to contact portion 2370A. Wire 2380A extends beyond the second end of sleeve 2374A. A first end of wire 2380A is aligned with proximal end 2360A of prong 2306A. Wire 2380A extends along base portion 2364A, arm portion 2368A, and contact portion 2370A. A second end of wire 2380A is connected to electrode 2372A. As such, contact portion 2370A of prong 2306A is made up of sleeve 2374A, wire 2380A, and electrode 2372A. Wire 2380A has the same overall shape and angle as sleeve 2374A and extends beyond the second end of sleeve 2374A. Wire 2380A extends away from the second end of sleeve 2374A and first side 2310A of housing 2302A. In this embodiment, wire 2380A extends about 90 degrees away from the second end of sleeve 2374A. As such, in this embodiment, wire 2380A is angled away from bottom side 2316A of housing 2302A along with sleeve 2374A and curved, or angled, away from first side 2310A of housing 2302A beyond sleeve 2374A.

Structural tubes 2382A and 2384A extend through sleeve 2374A, between upper portion 2376A and lower portion 2378A and along wire 2380A. Structural tubes 2382A and 2384A extend from proximal end 2360A of prong 2360A to the second end of arm portion 2368A. First ends of structural tubes 2382A and 2384A are aligned with proximal end 2360A of prong 2306A. Structural tubes 2382A and 2384A extend along base portion 2364A and arm portion 2368A. Second ends of structural tubes 2382A and 2384A are aligned with the second end of arm portion 2368A. In alternate embodiments, structural tubes 2382A and 2384A may extend into contact portion 2370A to the second end of sleeve 2374A such that second ends of structural tubes 2382A and 2384A are aligned with the second end of sleeve 2374A. Structural tubes 2382A and 2384A have the same overall shape as base portion 2364A and arm portion 2368A. As such, in this embodiment, structural tubes 2382A and 2384A are planar.

First structural tube 2382A is on a first side of wire 2380A, and second structural tube 2384A is on a second side of wire 2380A such that wire 2380A has structural tubes 2382A and 2384A on opposite sides of wire 2380A. In alternate embodiments, prong 2306A may include any number of structural tubes 2382A and 2384A based on the desired stiffness of prong 2306A. Structural tubes 2382A and 2384A may be hollow or solid. Structural tubes 2382A and 2384A can be any suitable size. For example, structural tubes 2382A and 2384A can have the same diameters as each other, can have the same diameter as wire 2380A, or can have a smaller diameter than wire 2380A. Structural tubes 2382A and 2384A can have any suitable thickness based on desired stiffness of prong 2306A. Structural tubes 2382A and 2384A may be made of metal, polyurethane, silicone, any suitable plastic, a combination of metal and plastic, or any other suitable material. Structural tubes 2382A and 2384A are limited to an amount of metal that allows subcutaneous device 2300 to be MRI compatible. In alternate embodiments, prong 2306A may include any number of structural tubes 2382A and 2384A. The size, shape, and material of structural tubes 2382A and 2384A may be selected based upon the desired stiffness of prong 2306A. For example, prong 2306A may include five, seven, or any other suitable number of structural tubes 2382A and 2384A to make prong 2306A flatter and increase the stiffness of prong 2306A.

Prongs 2306 and 2306A are angled with respect to housing 2302 to improve contact of electrodes 2372 and 2372A, respectively, with remote body component B. Prongs 2306 and 2306A are angled so that contact portions 2370 and 2370A push down against remote body component B, such as the heart. Because contact portion 2370A extends beyond contact portion 2370, electrode 2372A is positioned beyond electrode 2372. Electrode 2372 at distal end 2362 of prong 2306 and electrode 2372A at distal end 2362A of prong 2306A contact the heart and different locations and bury into the cardiac tissue. Further, because prongs 2306 and 2306A are angled down toward heart, prongs 2306 and 2306A apply pressure to the heart as the heart beats and moves up and down, without increasing the stiffness of prongs 2306 and 2306A. As a result, electrodes 2372 and 2372A maintain contact with the heart without fixing electrodes 2372 and 2372A to the heart. For example, prongs 2306 and 2306A are prevented from bouncing off of the heart as the heart beats, which would cause intermittent contact that reduces functionality. Because electrode 2372 is on prong 2306 and electrode 2372A is on separate prong 2306A, electrodes 2372 and 2372A both maintain contact with the heart even if the different locations of the heart on which electrodes 2372 and 2372A are positioned move asynchronously, such as in different directions or at different rates. Additionally, contact portions 2370 and 2370A are angled away from bottom 2316 and first side 2310 of housing 2302 to ensure distal ends 2362 and 2362A of prongs 2306 and 2306A, respectively, are positioned on the heart when subcutaneous device 2300 is attached to xiphoid process X and/or sternum S of a patient, as shown in FIG. 3D. As such, subcutaneous device 2300 can be inserted and deployed into a patient without requiring cardiac catheterization labs. Thus, the procedure for inserting the device is simple and only requires local anesthesia, which means it can be carried out in various environments, such as in an ambulance.

Arm portions 2368 and 2368A of prongs 2306 and 2306A allows prongs 2306 and 2306A, respectively, to be flexible once they are positioned in the body. The pivot points of arm portion 2368 and 2368A, respectively, are adjacent the first end of arm portion 2368 and 2368A, respectively, which are connected to the second ends of base portions 2364 and 2364A, respectively, and slightly closer to proximal ends 2360 and 2360A than front end 2318 of housing 2302 is to proximal ends 2306 and 2306A, or where prongs 2306 and 2306A are secured to bottom side 2316 of housing 2302 by housing latch 2322. For example, if remote body component B is the heart of the patient and contact portion 2370 of prong 2306 and contact portion 2370A of prong 2306A are positioned against the heart, arm portions 2368 and 2368A of prongs 2306 and 2306A, respectively, allow prongs 2306 and 2306 to move up and down with the heart as the heart beats. Further, prong 2306 can move separately from prong 2306A such that electrode 2372 and electrode 2372A both maintain contact with the heart. This ensures that prongs 2306 and 2306A do not puncture or damage the heart while contact portions 2370 and 2370A of prongs 2306 and 2306A, respectively, maintain contact with the heart. In this embodiment, electrodes 2372 and 2372A at distal ends 2362 and 2362A of prongs 2306 and 120 2306A, respectively, each have a rounded end to further prevent prongs 2306 and 2306A from puncturing or damaging the heart when contact portions 2370 and 2370A of prongs 2306 and 2360A, respectively, are in contact with the heart. The overall axial stiffness of prongs 2306 and 2306A can be adjusted so that prongs 2306 and 2306A gently presses against the heart and move up and down in contact with the heart, sometimes separately, as the heart beats, but are not stiff or sharp enough to pierce or tear the pericardial or epicardial tissue. For example, the overall axial stiffness of prongs 2306 and 2306A can be adjusted by adjusting the material of prongs 2306 and 2306A, the spring bias or mechanical resistance of prongs 2306 and 2306A, the cross-sectional thicknesses of prongs 2306 and 2306A, the angle of incidence of prongs 2306 and 2306A on remote body component B, the outer profiles of prongs 2306 and 2306A where prongs 2306 and 2306A contact remote body component B, and/or any other suitable characteristic of prongs 2306 and 2306A.

The flat, or rectangular, cross-section of sleeves 2374 and 2372A created by planar upper portions 2376 and 2376A and planar lower portions 2378 and 2378A provide stiffness to prongs 2306 and 2306A, respectively, which makes prongs 2306 and 2306A more resistant to in-plane bending. Sleeves 2374 and 2374A also provide space for wires 2380 and 2380A to be surrounded by structural tubes 2382 and 2382A and structural tubes 2384 and 2384A, respectively. Structural tubes 2382, 2382A, 2384, and 2384A also provide the desired structural stiffness to prongs 2306 and 2306A, respectively. As a result, prongs 2306 and 2306A resist in-plane bending, or bending in any direction, to maintain positioning with respect to the heart, which ensures electrode 2372 and electrode 2372A maintain contact with the heart without requiring fluoroscopy or other visualization tools. In alternate embodiments, prongs 2306 and 2306A may include pre-shaped spines made of shape-memory material, such as nitinol, to provide stiffness along with or instead of structural tubes 2282, 2284, 2282A, and 2284A. In these embodiments, prongs 2306 and 2306A may have the shape shown in FIG. 2A-2D, for example, or other suitable shapes or configurations.

Subcutaneous device 2300 is described here as having two prongs 2306 and 2306A. As such, electrodes 2372 and 2372A can contact different locations of remote body component B, such as the heart. In alternate embodiments, subcutaneous device 2300 can include prongs 2306 and 2306A that have any shape.

As shown in FIG. 3D, subcutaneous device 2300 can be anchored to xiphoid process X and sternum S of a patient. Subcutaneous device 2300 can be implanted with a simple procedure where subcutaneous device 2300 is injected onto xiphoid process X and sternum S using a surgical instrument. Subcutaneous device 2300 can function as a pacemaker. Prongs 2306 and 2306A can be shaped so that contact portion 2370 of prong 2306 and contact portion 2370A of prong 2306A contact the Manicka Zone of Pacing, as will be discussed in greater detail with respect to FIGS. 4A-5C. Subcutaneous device 2300 can function as a bipolar pacemaker, utilizing electrode 2372 on prong 2306 and electrode 2372A on prong 2306A.

### Subcutaneous Device 3000

FIG. 4A is a first oblique side view of subcutaneous device 3000 contacting heart H. FIG. 4B is a second oblique side view of subcutaneous device 3000 contacting heart H. For clarity and ease of discussion, FIGS. 4A-4B will be described together. Subcutaneous device 3000 includes housing 3002 and prong 3006. Prong 3006 includes electrode 3072A, electrode 3072B, wire 3080A, and wire 3080B. FIGS. 4A-4B also show heart H, including right ventricle RV, left ventricle LV, and Manicka Zone of Pacing MZP.

Subcutaneous device 3000 has the same general structure and design as subcutaneous device 2200 shown in FIGS. 2A-2D and subcutaneous device 2300 shown in FIGS. 3A-3D. Subcutaneous device 3000 is not shown as including a clip, but can include a clip in some embodiments. For ease of discussion, some components of subcutaneous device 3000 shown in FIGS. 4A-4B are not shown or described in detail in the following section, but it should be understood that subcutaneous device 3000 shown in FIGS. 4A-4B can include all or any combination of the components and features described with respect to FIGS. 2A-3D.

Subcutaneous device 3000 includes a single prong 3006 that has two electrodes, including electrode 3072A and electrode 3072B, and two wires, including wire 3080A and wire 3080B. In some embodiments, additional electrodes and wires could be included. Electrode 3072A is connected to wire 3080A, and electrode 3072B is connected to wire 3080B. Wire 3080A and wire 3080B both extend through prong 3006 and are electrically coupled to housing 3002. In the embodiment shown in FIGS. 4A-4B, wire 3080A extends in a first direction and wire 3080B extends in a second direction opposite of the first direction. In alternate embodiments, wire 3080A and wire 3080B can extend in the same direction or any other suitable directions with respect to one another. In the embodiment shown in FIGS. 4A-4B, subcutaneous device 3000 is configured to be a pacemaker used for cardiac monitoring, diagnostics, and/or therapeutics, such as subcutaneous device 2200 and 2300 as described regarding FIGS. 2A-3D.

Wire 3080A and wire 3080B are stiff wires with a stiffness sufficient to support electrode 3072A and electrode 3072B and hold electrode 3072A and electrode 3072B in position on heart H. In the embodiment shown in FIGS. 4A-4B, wire 3080A exits prong 3006 at a first position and wire 3080B exits prong 3006 at a second position that is longitudinally spaced from the first position. More specifically, wire 3080A exits prong 3006 at a side of prong 3006 and wire 3080B exits prong 3006 at a distal end of prong 3006. Alternatively, wire 3080A and wire 3080B can exit prong 3006 at the same position. Wire 3080A is angled at a 15 to 90 degree angle with respect to a longitudinal axis of prong 3006, and wire 3080B is angled at a 15 to 90 degree angle with respect to a longitudinal axis of prong 3006 on the opposite side of prong 3006 than wire 3080A.

In the embodiment shown in FIGS. 4A-4B, electrode 3072A and electrode 3072B are spaced laterally and longitudinally apart from one another. More specifically, electrode 3072A and electrode 3072B are spaced 1.5 to 6 centimeters (0.5906 to 2.3622 inches) from one another. Electrode 3072A is positioned 1 to 4 centimeters (0.3937 to 1.5748 inches) from a longitudinal axis of prong 3006, and electrode 3072B is positioned 1 to 4 centimeters (0.3937 to 1.5748 inches) from a longitudinal axis of prong 3006.

In the embodiment shown in FIGS. 4A-4B, subcutaneous device 3000 can be positioned on a xiphoid process and/or a sternum of a patient (not shown in FIGS. 4A-4B). When positioned on the xiphoid process and/or the sternum, prong 3006 extends toward and contacts heart H. Specifically, prong 3006 contacts heart H in Manicka Zone of Pacing MZP. In alternate embodiments, subcutaneous device 3000 can be positioned on any muscle, bone, or tissue that allows prong 3006 to contact Manicka Zone of Pacing MZP.

When subcutaneous device 3000 is anchored to a xiphoid process and/or a sternum via a clip, prong 3006 extends away from a first side and a bottom side of housing 3002. A contact portion of prong 3006 pushes down against heart H, and electrodes 3072A and 3072B at a distal end of prong 3006 contact heart H and maintains contact as heart H beats. Prong 3006 can be shaped so that prong 3006 contacts Manicka Zone of Pacing MZP of heart H. The overall desired stiffness of prong 3006 is achieved via structural tubes (as described with respect to FIGS. 2A-3D) or other structural components or materials within a sleeve of prong 3006, which ensures that prong 3006 gently presses against heart H and moves up and down in contact with heart H as heart H beats, but is not stiff or sharp enough to pierce or tear the pericardial or epicardial tissue.

Prong 3006 is shaped to ensure prong 3006 is properly positioned against and will not lose contact with heart H. The surgical procedure for implanting subcutaneous device 3000 is less invasive than the surgical procedure required for more traditional pacemaker devices, as subcutaneous device 3000 is placed subcutaneously in the body. No leads need to be positioned in the vasculature of the patient, lowering the risk of thrombosis to the patient.

FIG. 5A is a schematic depiction of subcutaneous device 3000 contacting heart H in a first position. FIG. 5B is a schematic depiction of subcutaneous device 3000 contacting heart H in a second position. FIG. 5C is a schematic depiction of subcutaneous device 3000 contacting heart H in a third position. For ease and clarity of discussion, FIGS. 5A-5C will be described together. Subcutaneous device 3000 includes prong 3006, which includes electrode 3072A, electrode 3072B, wire 3080A, and wire 3080B. FIGS. 5A-5C also show heart H, which includes right ventricle RV, left ventricle, LV, base B, apex AX, left anterior descending artery LADA, and Manicka Zone of Pacing MZP.

Subcutaneous device 3000 is described above in reference to FIGS. 4A-4B. Subcutaneous device 3000 is shown schematically in FIGS. 5A-5C.

Base B of heart H includes a region of right atrium RA, a region of left atrium LA, a region of interatrial septum, and a region of interventricular septum IS (shown in FIGS. 1A-1B). The general location of base B is shown as laterally extending dashed lines in FIGS. 5A-5C. In some examples, base B is a region that is generally between the atriums and ventricles.

Left anterior descending artery LADA is a branch of the left coronary artery of heart H that supplies the heart muscle with blood. It descends along heart H over interventricular septum IS (shown in FIG. 1B) on the anterior side of heart H. As described with respect to FIGS. 1A-1B, left anterior descending artery LADA is aligned with interventricular septum IS and is therefore aligned with bundle of His HIS, left bundle branches LBB, and right bundle branches RBB.

Manicka Zone of Pacing MZP is defined as an area of the heart extending 3 centimeters (1.1811 inches) laterally outward from either side of a left anterior descending artery of the heart. As such, Manicka Zone of Pacing MZP has a total lateral length of 6 centimeters (2.3633 inches). Electrode 3072A and electrode 3072B can contact heart H in an area extending longitudinally from a region superior to base B to apex AX of heart H within Manicka Zone of Pacing MZP. Electrode 3072A and electrode 3072B can contact heart H near base B of heart H within Manicka Zone of Pacing MZP. More specifically, electrode 3072A and electrode 3072B can contact heart H nearer base B than apex AX of heart H within Manicka Zone of Pacing MZP. Electrode 2072A and electrode 3072B can contact a pericardium or an epicardium of heart H within Manicka Zone of Pacing MZP. Because Manicka Zone of Pacing MZP includes left anterior descending artery LADA, and left anterior descending artery LADA is aligned with the interventricular septum, bundle of His HIS, left bundle branches LBB, and right bundle branches RBB (as described with respect to FIGS. 1A-1B), Manicka Zone of Pacing MZP is aligned with at least a portion of the native conductive pathway of heart H.

Prong 3006 extends towards heart H and includes electrode 3072A and electrode 3072B. Electrode 3072A is connected to wire 3080A, and electrode 3072B is connected to wire 3080B. This separates electrode 3072A and electrode 3072B from one another. For example, electrode 3072A can extend towards the right side of heart H (including right atrium RA and right ventricle RV) on a right side of left anterior descending artery LADA, and electrode 3072B can extend towards the left side of heart H (including left atrium LA and left ventricle LV) on a left side of left anterior descending artery LADA. In some examples, electrode 3072A and electrode 3072B share the same polarity. When subcutaneous device 3000 is connected to the xiphoid process and/or the sternum (as described with respect to FIGS. 2A-4B), prong 3006 is configured to extend to Manicka Zone of Pacing MZP, and electrode 3072A and electrode 3072B each contact heart H. In some embodiments, there can also be a third (center) electrode placed between electrodes 3072A and 3072B. The third electrode may have the same polarity or opposite polarity of electrodes 3072A and 3072B. Additional embodiments may include more electrodes between 3072A and 3072B. In one embodiment, each electrode is spaced 5 mm apart from each other, with the same or opposite polarity.

Referring now to FIG. 5A, prong 3006 is shown as extending towards base B of heart H. A distal end of prong 3006 is positioned within Manicka Zone of Pacing MZP. Accordingly, both electrode 3072A and electrode 3072B interact with heart H within Manicka Zone of Pacing MZP.

Electrode 3072A and electrode 3072B can each include a rounded end and have the shape described with respect to electrode 2272, electrode 2372, and/or electrode 2372A described in FIGS. 2A-3D. In alternate embodiments, electrode 3072A and electrode 3072B may have any suitable shape. Electrode 3072A and electrode 3072B each contact heart H to sense an electrical activity or physiological status of heart H and provide therapeutic electrical stimulation to heart H.

In some examples, electrode 3072A and/or 3072B sense an electrical activity or physiological status of heart H and deliver a therapeutic electrical signal (pacing signal) to a respective contact point within Manicka Zone of Pacing MZP. In one example, wire 3080A extends to the right of left anterior descending artery LADA, and electrode 3072A has a contact point within Manicka Zone of Pacing MZP to the right of left anterior descending artery LADA and along base B of heart H (as shown in FIG. 5A). Wire 3080B extends to the left of left anterior descending artery LADA, and electrode 3072B has a contact point within Manicka Zone of Pacing MZP to the left of left anterior descending artery LADA and along base B of heart H (as shown in FIG. 5A). In some examples, electrode 3072A has a contact point that is aligned with bundle of His HIS (shown in FIG. 1B) on the anterior surface of heart H. Electrode 3072A and electrode 3072B can deliver therapeutic electrical signals to bundle of His HIS or the basal portion of left bundle branch LBB and/or right bundle branch RBB. The therapeutic electrical signals can propagate from the anterior surface of heart H to 9 millimeters to 11 millimeters or more through the surface of heart H to reach bundle of His HIS, left bundle branch LBB, and/or right bundle branch RBB. The therapeutic electric signals are transmitted through the native conduction system of heart H, such as by bundle of His HIS and/or by left bundle branch LBB and/or right bundle branch RBB. Bundle of His HIS relays the therapeutic signals to right bundle branch RBB and left bundle branch LBB. Right bundle branch RBB and left bundle branch LBB relay the therapeutic signals to Purkinje fibers PF to synchronously contract right ventricle RV and left ventricle LV.

In another example, neither electrode 3072A nor electrode 3072B have a contact point aligned with bundle of His HIS. In such an example, the therapeutic electrical signal can still propagate to bundle of His HIS and cause right ventricle RV and left ventricle LV to contract in the same way as discussed above.

In another example, neither electrode 3072A nor electrode 3072B have a contact point aligned with either bundle of His HIS or base B of heart H. In such an example, the therapeutic electrical signal can propagate from the anterior surface of heart H to 9 millimeters to 11 millimeters or more through the surface of heart H to reach right ventricle RV and left ventricle LV. For example, electrode 3072A can deliver the therapeutic signal to right ventricle RV, and electrode 3072B can deliver the therapeutic signal to left ventricle LV. The electrical signals directly cause, respectively, right ventricle RV and left ventricle LV to contract simultaneously. Since the electrode stimulation location in Manicka Zone of Pacing MZP is on the anterior surface of heart H near left anterior descending artery LADA, and because left anterior descending artery LADA is associated with the interventricular septum between left ventricle LV and right ventricle RV, the stimulation point occurs approximately symmetric and equidistant from left ventricle LV and right ventricle RV. As a consequence, the pacing contraction will proceed symmetrically and simultaneously on both left ventricle LV and right ventricle RV, resulting in a synchronous muscular contraction of both left ventricle LV and right ventricle RV.

While the ventricle contraction examples provided with respect to FIG. 5A are described as separate examples, it should be understood that the contraction of right ventricle RV and left ventricle LV can occur due to one or any combination of the examples described.

Referring now to FIG. 5B, prong 3006 is shown as extending towards base B of heart H. Wire 3080A extends outside of Manicka Zone of Pacing MZP, and wire 3080B extends within Manicka Zone of Pacing MZP. Accordingly, electrode 3072A interacts with heart H at a region outside of Manicka Zone of Pacing MZP, and electrode 3072B interacts with H at a region within Manicka Zone of Pacing MZP.

Electrode 3072A and electrode 3072B can each include a rounded end and have the shape described with respect to electrode 2272, electrode 2372, and/or electrode 2372A described in FIGS. 2A-3D. In alternate embodiments, electrode 3472 and electrode 3072B may have any suitable shape. Electrode 3072A and electrode 3072B contact heart H to sense an electrical activity or physiological status of heart H and provide therapeutic electrical stimulation to heart H.

In some examples, electrode 3072A and electrode 3072B sense an electrical activity or physiological status of heart H and deliver therapeutic electrical signals to heart H. Electrode 3072A delivers a therapeutic electrical signal to a contact point outside of Manicka Zone of Pacing MZP, and electrode 3072B delivers a therapeutic electrical signal to a contact point within Manicka Zone of Pacing MZP. In one example, the contact point of electrode 3072B within Manicka Zone of Pacing MZP is to the right of left anterior descending artery LADA and along base B of heart H (as shown in FIG. 5B). The contact point can be aligned with bundle of His HIS. Electrode 3072B can deliver a therapeutic signal to the contact point, and the signal can propagate from the anterior surface of heart H to 9 millimeters to 11 millimeters or more through the surface of heart H to reach bundle of His HIS, left bundle branch LBB, or right bundle branch RBB. The therapeutic electric signal is transmitted through the native conductive system of heart H, such as by bundle of His HIS, left bundle branch LBB, and/or right bundle branch RBB. Bundle of His HIS relays the therapeutic signal to right bundle branch RBB and left bundle branch LBB. Right bundle branch RBB and left bundle branch LBB relay the therapeutic signal to Purkinje fibers PF to synchronously contract right ventricle RV and left ventricle LV.

In another example, the contact point of electrode 3072B is not aligned with bundle of His HIS. In such an example, the therapeutic electrical signal can still propagate to bundle of His HIS and cause right ventricle RV and left ventricle LV to contract in the same way as discussed above.

In another example, the contact point of electrode 3072B is not aligned with bundle of His HIS and doesn't propagate to bundle of His HIS. In such an example, the therapeutic electrical signal can still propagate from the anterior surface of heart H to 9 millimeters to 11 millimeters or more through the surface of H to directly reach to left bundle branch LBB or right bundle branch RBB. This electrical signals directly causes right ventricle RV and left ventricle LV to contract.

In yet another example, the contact point of electrode 3072B is not aligned with either bundle of His HIS or base B of heart H. In such an example, the therapeutic electrical signal can propagate from the anterior surface of heart H to 9 millimeters to 11 millimeters or more through the surface of heart H to reach the myocardial tissue of right ventricle RV and left ventricle LV. This electrical signal directly causes right ventricle RV and left ventricle LV to contract without using the native conductive system of heart H.

While the ventricle contraction examples provided with respect to FIG. 5B are described as separate examples, it should be understood that the contraction of right ventricle RV and left ventricle LV can occur due to one or any combination of the examples described.

Referring now to FIG. 5C, prong 3006 is shown as extending towards base B of heart H. Wire 3080A extends within Manicka Zone of Pacing MZP, and wire 3080B extends outside of Manicka Zone of Pacing MZP. Accordingly, electrode 3072A interacts with heart H at a region within Manicka Zone of Pacing MZP, and electrode 3072B interacts with H at a region outside of Manicka Zone of Pacing MZP.

Electrode 3072A and electrode 3072B can each include a rounded end and have the shape described with respect to electrode 2272, electrode 2372, and/or electrode 2372A described in FIGA. 2A-3D. In alternate embodiments, electrode 3072A and electrode 3072B may have any suitable shape. Electrode 3072A and electrode 3072B contact heart H to sense an electrical activity or physiological status of heart Hand provide therapeutic electrical stimulation to heart H.

In some examples, electrode 3072A and electrode 3072B sense an electrical activity or physiological status of heart H and deliver therapeutic electrical signals to heart H. Electrode 3072A delivers a therapeutic electrical signal to a contact point within Manicka Zone of Pacing MZP, and electrode 3072B delivers a therapeutic electrical signal to a contact point outside of Manicka Zone of Pacing MZP. In one example, the contact point of electrode 3072A within Manicka Zone of Pacing MZP is to the left of left anterior descending artery LADA and along base B of heart H (as shown in FIG. 5C). The contact point can be aligned with bundle of His HIS. Electrode 3072A can deliver a therapeutic signal to the contact point, and the signal can propagate from the anterior surface of heart H to 9 millimeters to 11 millimeters or more through the surface of heart H to reach bundle of His HIS, left bundle branch LBB, or right bundle branch RBB. The therapeutic electric signal is transmitted through the native conductive system of heart H, such as by bundle of His HIS, left bundle branch LBB, and/or right bundle branch RBB. Bundle of His HIS relays the therapeutic signal to right bundle branch RBB and left bundle branch LBB. Right bundle branch RBB and left bundle branch LBB relay the therapeutic signal to Purkinje fibers PF to synchronously contract right ventricle RV and left ventricle LV.

In another example, the contact point of electrode 3072A is not aligned with bundle of His HIS. In such an example, the therapeutic electrical signal can still propagate to bundle of His HIS and cause right ventricle RV and left ventricle LV to contract in the same way as discussed above.

In another example, the contact point of electrode 3072A is not aligned with bundle of His HIS and doesn't propagate to bundle of His HIS. In such an example, the therapeutic electrical signal can still propagate from the anterior surface of heart H to 9 millimeters to 11 millimeters or more through the surface of H to directly reach to left bundle branch LBB or right bundle branch RBB. This electrical signals directly causes right ventricle RV and left ventricle LV to contract.

In yet another example, the contact point of electrode 3072A is not aligned with either bundle of His HIS or base B of heart H. In such an example, the therapeutic electrical signal can propagate from the anterior surface of heart H to 9 millimeters to 11 millimeters or more through the surface of heart H to reach the myocardial tissue of right ventricle RV and left ventricle LV. This electrical signal directly causes right ventricle RV and left ventricle LV to contract without using the native conductive system of heart H.

While the ventricle contraction examples provided with respect to FIG. 5C are described as separate examples, it should be understood that the contraction of right ventricle RV and left ventricle LV can occur due to one or any combination of the examples described.

The double electrode configuration of subcutaneous device 3000 of FIGS. 5A-5C (or the three electrode configuration of subcutaneous device 3100 shown in FIGS. 6-7) makes the native conductive system of heart H accessible to therapeutic intervention without requiring expensive and complex medical procedures. It is also possible for any one of the electrodes to individually activate Bundle of His HIS, left bundle branch LBB, right bundle branch RBB, or the ventricular myocardium individually on their own, without the need for the other electrode(s). Thus, it is possible to achieve ventricular synchrony (V-V synchrony) with pacing with just one electrode placed in Manicka Zone of Pacing MZP. The current embodiment describes pacing with one-, two-, three- or more electrodes, at least one of the electrodes placed within the Manicka Zone of Pacing to achieve ventricular synchrony (V-V synchrony). For example, with this configuration, accessing bundle of His HIS, left bundle branch LBB, or right bundle branch RBB does not require an invasive procedure of drilling through the septum, and no procedurally complicated imaging is required to find bundle of His HIS, left bundle branch LBB, or right bundle branch RBB. Therapeutic intervention without additional complication is possible because anchoring the subcutaneous device onto a xiphoid process and/or a sternum naturally positions electrode 3072A and electrode 3072B to be electrically connected to Manicka Zone of Pacing MZP, and specifically bundle of His HIS, right bundle branch RBB, left bundle branch LBB, and/or right ventricle RV and left ventricle LV. This configuration permits right ventricle RV and left ventricle LV to synchronously receive therapeutic electrical stimulation without necessitating precise placement of electrode 3072A or electrode 3072B. The position of electrode 3072A and/or electrode 3072B within Manicka Zone of Pacing MZP further results in delivery of therapeutic electrical stimulation synchronously to right ventricle RV and left ventricle LV. This reduces the time typically required to propagate an electrical signal from one ventricle to another associated with traditional pacing devices, thereby reducing the early onset of dilated myocardiopathy. Accordingly, subcutaneous device 3000 is an improvement over traditional subcutaneous pacing devices, including right ventricular pacing devices and biventricular pacing devices.

Subcutaneous device 3000 is one example of a device that can be used to pace Manicka Zone of Pacing MZP. Manicka Zone of Pacing MZP can also be paced using any other device having electrodes that are configured to contact Manicka Zone of Pacing MZP on the pericardium or an epicardium of heart H.

Pacing in the Manicka Zone of Pacing MZP allows for synchronized pacing of heart H while also simplifying the surgical procedure for implanting a device for pacing the heart. Subcutaneous device 3000 is implanted using a subcutaneous approach. No leads have to be extended through the patient's vasculature, simplifying the surgical procedure and minimizing risk to the patient.

Pacing heart H on the pericardium of heart H in Manicka Zone of Pacing MZP (near the intersection of left anterior descending artery LADA and base B of heart H) is an improved treatment. Left anterior descending artery LADA is associated with interventricular septum IS. Subcutaneous device 3000 is designed to target right ventricle RV and left ventricle LV very close to left anterior descending artery LADA and base B of heart H. Electrodes 3072A and 3072B will rest on the pericardium of heart H at locations immediately anterior to interventricular septum IS around bundle of His HIS, right bundle branch RBB, and left bundle branch LBB. There is no anatomical separation of cardiac tissue into chambers or septum on the anterior surface of heart H. Electrodes 3072A and 3072B will be positioned near left anterior descending artery LADA and also very close to interventricular septum IS on the anterior surface of heart H. The electrical signal from electrodes 3072A and/or 3072B will reach left ventricle LV very quickly. The pacing impulse is transmitted by direct contraction of cardiac myocytes and muscle tissue in addition to conduction through right bundle branch RBB and left bundle branch LBB. This mechanism activates contraction of left ventricle LV with a minimal delay, similar to left bundle branch pacing.

Pacing with at least one electrode in the Manicka Zone of Pacing MZP is defined as Manicka Synchronous Pacing (MSP). Manicka Synchronous Pacing results in synchronous pacing of both right ventricle RV and left ventricle LV with a minimal interventricular delay. The response to Manicka Synchronous Pacing in the Manicka Zone of Pacing MZP is similar to that of left bundle branch pacing, bundle of His pacing, or biventricular pacing without the complexity of implanting one or more leads in the vasculature of the patient. Manicka Synchronous Pacing in the Manicka Zone of Pacing MZP minimizes the complexity of the implant procedure, minimizes risk, and improves safety. Further, implanting subcutaneous device 3000 to pace in the Manicka Zone of Pacing MZP may not require imaging to implant subcutaneous device 3000.

Manicka Synchronous Pacing in the Manicka Zone of Pacing MZP allows for contemporaneous electrical activation of anterior right bundle branch RBB and left bundle branch LBB and the interventricular surface anterior to interventricular septum IS. Pacing in the Manicka Zone of Pacing is an effective physiological substitute with much less complexity compared to right ventricular pacing, biventricular pacing, bundle of His pacing, left bundle branch pacing, and any other pacing using a coronary sinus lead and optimizing the interventricular pacing delay.

Pacing thresholds when pacing in Manicka Zone of Pacing MZP will likely be higher than pacing thresholds for traditional endocardial leads because pacing is achieved from outside the pericardium. The pacing thresholds when pacing in Manicka Zone of Pacing MZP may also be also higher than thresholds for leads positioned in the coronary sinus. Pacing through the adipose tissue surrounding left anterior descending artery LADA also allows for increased pacing thresholds when pacing in Manicka Zone of Pacing MZP. In many cases the Manicka Synchronous Pacing is delivered from subcutaneous device 3000 subcutaneously, which may be designed to be rechargeable, allowing for higher pacing thresholds without concern about the life/duration of subcutaneous device 3000.

Manicka Synchronous Pacing in Manicka Zone of Pacing MZP using subcutaneous device 3000 or any other similar device prevents perforation of the pericardium, myocardium, septum, or any other cardiac tissue. Further, no hardware is implanted inside heart H, reducing risk to the patient. Subcutaneous device 3000 does not require any leads be placed in the vasculature of the patient. Subcutaneous device 3000 is attached to the skeletal structure of the patient (including any muscle, bone, or tissue). In one example, subcutaneous device 3000 can be anchored to the xiphoid process and/or the sternum of the patient and prong 3006 can be placed in-between natural muscular folds to reach heart H.

### Subcutaneous Device 3100

FIG. 6 is a perspective of subcutaneous device 3100. FIG. 7 is a schematic depiction of subcutaneous device 3100 contacting heart H in a first position. For clarity and ease of discussion, FIGS. 6-7 will be described together. Subcutaneous device 3100 includes housing 3102 and prong 3106. Prong 3106 includes electrode 3172A, electrode 3172B, electrode 3172C, wire 3180A, wire 3180B, and wire 3180C. FIG. 7 also shows heart H, including right ventricle RV, left ventricle LV, and Manicka Zone of Pacing MZP.

Subcutaneous device 3100 has the same general structure and design as subcutaneous device 2200 shown in FIGS. 2A-2D, subcutaneous device 2300 shown in FIGS. 3A-3D, and subcutaneous device 3000 shown in FIGS. 4A-5C. Subcutaneous device 3100 is not shown as including a clip, but can include a clip in some embodiments. For ease of discussion, some components of subcutaneous device 3100 shown in FIGS. 6-7 are not shown or described in detail in the following section, but it should be understood that subcutaneous device 3100 shown in FIGS. 6-7 can include all or any combination of the components and features described with respect to FIGS. 2A-3D.

Subcutaneous device 3100 includes a single prong 3106 that has three electrodes, including electrode 3172A, electrode 3172B, and electrode 3272C, and three wires, including wire 3180A, wire 3180B, and wire 3180C. In some embodiments, additional electrodes and wires could be included. Electrode 3172A is connected to wire 3180A, electrode 3172B is connected to wire 3180B, and electrode 3172C is connected to wire 3180C. Wire 3180A, wire 3180B, and wire 3180C extend through prong 3106 and are electrically coupled to housing 3102. In the embodiment shown in FIGS. 6-7, wire 3180A extends in a first direction, wire 3180B extends in a second direction, and wire 3180C extends in a third direction. The first direction is opposite the third direction. In the embodiment shown in FIGS. 6-7, the first direction is away from a longitudinal axis of prong 3106 on a first side, the third direction is away from the longitudinal axis of prong 3106 on a second side, and the second direction is generally aligned with the longitudinal axis of prong 3106. In alternate embodiments, wire 3180A, wire 3180B, and wire 3180C can extend in the same directions or any other suitable directions with respect to one another. In the embodiment shown in FIGS. 6-7, subcutaneous device 3100 is configured to be a pacemaker used for cardiac monitoring, diagnostics, and/or therapeutics, such as subcutaneous device 2200 and 2300 as described regarding FIGS. 2A-3D.

Wire 3180A, wire 3180B, and 3108C are stiff wires with a stiffness sufficient to support electrode 3172A, electrode 3172B, and electrode 3172C and hold electrode 3172A, electrode 3172B, and electrode 3172C in position on heart H. In the embodiment shown in FIGS. 6-7, wire 3180A, wire 3180B, and wire 3180C exit prong 3106 at the same position. In alternative embodiments, wire 3180A, wire 3180B, and wire 3180C exit prong 3106 are different positions along the length of prong 3106. Wire 3180A is angled at a 15 to 90 degree angle with respect to a longitudinal axis of prong 3106, and wire 3180C is angled at a 15 to 90 degree angle with respect to the longitudinal axis of prong 3106 on the opposite side of prong 3106 than wire 3180A.

In the embodiment shown in FIGS. 6-7, electrode 3172A, electrode 3172B, and electrode 3172C are spaced laterally and longitudinally apart from one another. More specifically, electrode 3172A and electrode 3172C are spaced 1.5 to 6 centimeters (0.5906 to 2.3622 inches) from one another. Electrode 3172A is positioned 1 to 4 centimeters (0.3937 to 1.5748 inches) from a longitudinal axis of prong 3106, and electrode 3172C is positioned 1 to 4 centimeters (0.3937 to 1.5748 inches) from a longitudinal axis of prong 3106.

In the embodiment shown in FIGS. 6-7, subcutaneous device 3100 can be positioned on a xiphoid process and/or a sternum of a patient (not shown in FIGS. 6-7). When positioned on the xiphoid process and/or the sternum, prong 3106 extends toward and contacts heart H. Specifically, prong 3106 contacts heart H in Manicka Zone of Pacing MZP. In alternate embodiments, subcutaneous device 3100 can be positioned on any muscle, bone, or tissue that allows prong 3106 to contact Manicka Zone of Pacing MZP.

When subcutaneous device 3100 is anchored to a xiphoid process and/or a sternum via a clip, prong 3106 extends away from a first side and a bottom side of housing 3102. A contact portion of prong 3106 pushes down against heart H, and electrodes 3172A, electrode 3172B, and electrode 3172C at a distal end of prong 3106 contact heart H and maintain contact as heart H beats. Prong 3106 can be shaped so that prong 3106 contacts Manicka Zone of Pacing MZP of heart H. The overall desired stiffness of prong 3106 is achieved via structural tubes (as described with respect to FIGS. 2A-3D) or other structural components or materials within a sleeve of prong 3106, which ensures that prong 3106 gently presses against heart H and moves up and down in contact with heart H as heart H beats, but is not stiff or sharp enough to pierce or tear the pericardial or epicardial tissue.

Prong 3106 is shaped to ensure prong 3106 is properly positioned against and will not lose contact with heart H. The surgical procedure for implanting subcutaneous device 3100 is less invasive than the surgical procedure required for more traditional pacemaker devices, as subcutaneous device 3100 is placed subcutaneously in the body. No leads need to be positioned in the vasculature of the patient, lowering the risk of thrombosis to the patient.

Subcutaneous device 3100 can be used to pace Manicka Zone of Pacing MZP, as discussed above in reference to FIGS. 4A-5C. As shown in FIG. 7, all three electrodes, including electrode 3172A, electrode 3172B, and electrode 3172C of subcutaneous device 3100 can contact heart H in Manicka Zone of Pacing MZP. In this example, electrode 3172A can extend toward the right side of heart H (including right atrium RA and right ventricle RV) on a right side of left anterior descending artery LADA, electrode 3172C can extend toward the left side of heart H (including left atrium LA and left ventricle LV) on a left side of left anterior descending artery LADA, and electrode 3172B can extend generally along left anterior descending artery LADA. In some examples, electrode 3172A, electrode 3172B, and electrode 3172C share the same polarity or have different polarities. For example, electrode 3172A and electrode 3172C can share the same polarity, and electrode 3172B can have an opposite polarity. In one embodiment, each electrode is spaced 5 mm apart from each other, with the same or opposite polarity.

Alternatively, only electrode 3172A and electrode 3172B can contact heart H in Manicka Zone of Pacing MZP, and electrode 3172C can contact heart H outside of Manicka Zone of Pacing MZP. For example, electrode 3172A can contact heart H along left anterior descending artery, electrode 3172B can contact heart H to the left side of left anterior descending artery LADA within Manicka Zone of Pacing MZP, and electrode 3172C can contact heart H to the left side of left anterior descending artery LADA and outside of Manicka Zone of Pacing MZP.

Alternatively, only electrode 3172B and electrode 3172C can contact heart H in Manicka Zone of Pacing MZP, and electrode 3172A can contact heart H outside of Manicka Zone of Pacing MZP. For example, electrode 3172C can contact heart H along left anterior descending artery, electrode 3172B can contact heart H to the right side of left anterior descending artery LADA within Manicka Zone of Pacing MZP, and electrode 3172A can contact heart H to the right side of left anterior descending artery LADA and outside of Manicka Zone of Pacing MZP.

Alternatively, only electrode 3172A can contact heart H in Manicka Zone of Pacing MZP, and electrode 3172B and electrode 3172C can contact heart H outside of Manicka Zone of Pacing MZP. For example, electrode 3172A can contact heart H to the left side of left anterior descending artery LADA within Manicka Zone of Pacing MZP, and electrode 3172B and electrode 3172C can contact heart H to the left side of left anterior descending artery LADA and outside of Manicka Zone of Pacing MZP.

Alternatively, only electrode 3172C can contact heart H in Manicka Zone of Pacing MZP, and electrode 3172A and 3172B can contact heart H outside of Manicka Zone of Pacing MZP. For example, electrode 3172C can contact heart H to the right side of left anterior descending artery LADA within Manicka Zone of Pacing MZP, and electrode 3172A and electrode 3172B can contact heart H to the right side of left anterior descending artery LADA and outside of Manicka Zone of Pacing MZP.

As long as one of electrode 3172A, electrode 3172B, and electrode 3172C contact heart H in Manicka Zone of Pacing MZP, subcutaneous device 3100 can synchronously pace right ventricle RV and left ventricle LV of heart H. Further, if two or more of electrode 3172A, electrode 3172B, and electrode 3172C contact heart H in Manicka Zone of Pacing MZP, subcutaneous device 3100 can also synchronously pace right ventricle RV and left ventricle LV of heart H. Additionally, if all three of electrode 3172A, electrode 3172B, and electrode 3172C contact heart H in Manicka Zone of Pacing MZP, subcutaneous device 3100 can also synchronously pace right ventricle RV and left ventricle LV and heart H.

As described above with respect to FIGS. 5A-5C, electrode 3172A, electrode 3172B, and/or electrode 3172C can be aligned with a portion of a native conductive pathway of heart H when they contact heart H in Manicka Zone of Pacing MZP. More specifically, electrode 3172A, electrode 3172B, and/or electrode 3172C can be aligned with interventricular septum IS, bundle of His HIS, left bundle branch LBB, and/or right bundle branch RBB when they contact heart H in Manicka Zone of Pacing MZP.

In some examples, electrode 3172A, electrode 3172B, and/or electrode 3172C sense an electrical activity or physiological status of heart H and deliver a therapeutic electrical signal (pacing signal) to a respective contact point within Manicka Zone of Pacing MZP. As described above with respect to FIGS. 5A-5C, any of electrode 3172A, electrode 3172B, and/or electrode 3172C that contact heart H in Manicka Zone of Pacing MZP can deliver an electrical signal to bundle of His HIS, left bundle branch LBB, right bundle branch RBB, Purkinje fibers PF, and/or the myocardial tissue of left ventricle LV and right ventricle RV.

The three electrode configuration of subcutaneous device 3100 of FIGS. 6-7 makes the native conductive system of heart H accessible to therapeutic intervention without requiring expensive and complex medical procedures. It is also possible for any one of the electrodes to individually activate bundle of His HIS, left bundle branch LBB, right bundle branch RBB, or the ventricular myocardium individually on their own, without the need for the other electrode(s). Thus, it is possible to achieve ventricular synchrony (V-V synchrony) with pacing using just one electrode placed in Manicka Zone of Pacing MZP. The current embodiment describes pacing with one-, two-, three- or more electrodes, at least one of the electrodes placed within the Manicka Zone of Pacing to achieve ventricular synchrony (V-V synchrony). For example, with this configuration, accessing bundle of His HIS, left bundle branch LBB, or right bundle branch RBB does not require an invasive procedure of drilling through the septum, and no procedurally complicated imaging is required to find bundle of His HIS, left bundle branch LBB, or right bundle branch RBB. Therapeutic intervention without additional complication is possible because anchoring the subcutaneous device onto a xiphoid process and/or a sternum naturally positions electrode 3172A, electrode 3172B, and/or electrode 3172C to be electrically connected to Manicka Zone of Pacing MZP, and specifically bundle of His HIS, right bundle branch RBB, left bundle branch LBB, and/or right ventricle RV and left ventricle LV. This configuration permits right ventricle RV and left ventricle LV to synchronously receive therapeutic electrical stimulation without necessitating precise placement of electrode 3172A, electrode 3172B, and/or electrode 3172C. The position of electrode 3172A, electrode 3172B, and/or electrode 3173C within Manicka Zone of Pacing MZP further results in delivery of therapeutic electrical stimulation synchronously to right ventricle RV and left ventricle LV. This reduces the time typically required to propagate an electrical signal from one ventricle to another associated with traditional pacing devices, thereby reducing the early onset of dilated myocardiopathy. Accordingly, subcutaneous device 3100 is an improvement over traditional subcutaneous pacing devices, including right ventricular pacing devices and biventricular pacing devices.

### Electrocardiogram Graphs

The effectiveness of pacing in Manicka Zone of Pacing MZP can be demonstrated by the width of the QRS complex of heart H. The width of the QRS complex for right ventricular pacing and biventricular pacing is wider than the natural QRS complex of normal, healthy hearts, which is caused due to the electrical and mechanical dyssynchrony of the heart (as discussed above in reference to FIGS. 1A-1B). In comparison, the width of the QRS complex for bundle of His pacing and left bundle branch pacing is narrower than the width of the QRS complex for right ventricular pacing and biventricular pacing but wider than the natural QRS complex of normal, healthy hearts. The width of the QRS complex in Manicka Zone of Pacing MZP is also typically somewhat wider than the natural QRS complex of normal, healthy hearts, but may be similar to, or slightly narrower than, the width of the QRS complex with bundle of His pacing or left bundle branch pacing.

FIG. 8A is an electrocardiogram graph showing a QRS complex for a normal, healthy heart. FIG. 8B is a first electrocardiogram graph showing a QRS complex when pacing a Manicka Zone of Pacing. FIG. 8C is a second electrocardiograph graph showing a QRS complex when pacing the Manicka Zone of Pacing. FIG. 8A shows QRS complex QRS1 and width W1. FIG. 8B shows QRS complex QRS2 and width W2. FIG. 8C shows QRS complex QRS3 and width W3.

FIGS. 8A-8C show electrocardiogram graphs generated in tests using a Swine model. FIG. 8A shows multiple QRS complexes for a healthy, normal heart. One QRS complex QRS1 is labeled in FIG. 8A for simplicity. QRS complex QRS1 had width W1, which represents an average width for the QRS complexes shown in FIG. 8A. Width W1 can be approximately 57.8 milliseconds.

FIG. 8B shows multiple QRS complexes for a heart that is being paced in the Manicka Zone of Pacing. For example, the heart can be paced in the Manicka Zone of Pacing using subcutaneous device 3000 shown in FIGS. 4A-5C or subcutaneous device 3100 shown in FIGS. 6-7. One QRS complex QRS2 is labeled in FIG. 8B for simplicity. QRS complex QRS2 has width W2, which represents an average width for the QRS complexes shown in FIG. 8B. Width W2 can be approximately 80.9 milliseconds.

FIG. 8C shows multiple QRS complexes for a heart that is being paced in the Manicka Zone of Pacing. For example, the heart can be paced in the Manicka Zone of Pacing using subcutaneous device 3000 shown in FIGS. 4A-5C or subcutaneous device 3100 shown in FIGS. 6-7. One QRS complex QRS3 is labeled in FIG. 8C for simplicity. QRS complex QRS3 has width W3, which represents an average width for the QRS complexes shown in FIG. 8C. Width W3 can be approximately 82.6 milliseconds.

The electrocardiogram graphs shown in FIGS. 8A-8C demonstrate the efficacy of pacing in the Manicka Zone of Pacing. The width of QRS complexes using other pacing therapies is typically between 100 milliseconds and 160 milliseconds. Pacing in the Manicka Zone of Pacing allows for the width of QRS complexes to be narrower than using other pacing therapies. The narrower width of QRS complexes when pacing in the Manicka Zone of Pacing indicates that the right ventricle and left ventricle are being synchronously paced.

Manicka Synchronous Pacing in the Manicka Zone of Pacing MZP has been discussed above in an embodiment of a pacemaker inserted under the xiphoid process/inferior sternum. Other embodiments of the concept include traditional pectoral/abdominal pacemakers connected to subcutaneous flexible leads in which at least one of the pacing electrodes are placed on the anterior surface of the heart in the Manicka Zone of Pacing MZP. When traditional pacemakers are used to pace Manicka Zone of Pacing MZP, the lead(s) can be tunneled through the pericardium and into the pericardial cavity. The lead(s) can then be positioned so that the electrode(s) on the lead(s) are positioned on the epicardium in the Manicka Zone of Pacing MZP. As such, traditional pacemakers can be used to pace heart H in Manicka Zone of Pacing MZP. Traditional pacemaker devices can include cardiac resynchronization therapy (CRT) devices and implantable cardioverter-defibrillator (ICD) devices.

### Discussion of Possible Embodiments

The following are non-exclusive descriptions of possible embodiments of the present invention.

A method of pacing a heart includes implanting a subcutaneously implantable device on a xiphoid process and/or a sternum of a patient. The subcutaneously implantable device includes a housing, a prong extending away from the housing, and a first electrode at a distal end of the prong. The method further includes contacting a Manicka Zone of Pacing on an anterior surface of the heart with the first electrode on the distal end of the prong. The Manicka Zone of Pacing is defined as an area of the heart extending 3 centimeters (1.1811 inches) laterally outward from either side of a left anterior descending artery of the heart. A pacing signal is delivered to the Manicka Zone of Pacing of the heart through the first electrode. A right ventricle and a left ventricle of the heart are synchronously paced with the pacing signal received in the Manicka Zone of Pacing.

The method of the preceding paragraph can optionally include, additionally and/or alternatively, any one or more of the following features, configurations and/or additional components:

Wherein the first electrode contacts the heart in an area extending longitudinally from a region superior to the base of the heart to an apex of the heart within the Manicka Zone of Pacing.

Wherein the first electrode contacts the heart near the base of the heart within the Manicka Zone of Pacing.

The method further includes aligning the first electrode with a portion of a native conductive pathway of the heart.

The method further includes aligning the first electrode contacting the anterior surface of the heart with an interventricular septum, a bundle of His, a left bundle branch, and/or a right bundle branch.

Wherein delivering the pacing signal to the Manicka Zone of Pacing of the heart through the first electrode further includes synchronously delivering the pacing signal to the right ventricle and the left ventricle of the heart.

The method further includes delivering the pacing signal to the bundle of His.

The method further includes delivering the pacing signal to the bundle of His, the left bundle branch, the right bundle branch, and/or the Purkinje fibers.

Wherein contacting the Manicka Zone of Pacing of the heart with the first electrode on the distal end of the prong further includes contacting a pericardium or an epicardium of the heart in the Manicka Zone of Pacing of the heart with the first electrode on the distal end of the prong.

The method further includes actively fixing the first electrode in the myocardial tissue in the Manicka Zone of Pacing of the heart.

A method of pacing a heart includes implanting a subcutaneously implantable device on a xiphoid process and/or a sternum of a patient. The subcutaneously implantable device includes a housing, a prong extending away from the housing, and a first electrode and a second electrode at a distal end of the prong. The method further includes contacting a Manicka Zone of Pacing of the heart with the first electrode and/or the second electrode on the distal end of the prong. The Manicka Zone of Pacing is defined as an area of the heart extending 3 centimeters (1.1811 inches) laterally outward from either side of a left anterior descending artery of the heart. The first electrode and/or the second electrode contact the heart near the base of the heart within the Manicka Zone of Pacing. A pacing signal is delivered to the Manicka Zone of Pacing of the heart through the first electrode and/or the second electrode. The right ventricle and a left ventricle of the heart are synchronously paced with the pacing signal received in the Manicka Zone of Pacing.

The method of the preceding paragraph can optionally include, additionally and/or alternatively, any one or more of the following features, configurations and/or additional components:

Wherein contacting the Manicka Zone of Pacing of the heart with the first electrode and/or the second electrode on the distal end of the prong further includes contacting the Manicka Zone of Pacing of the heart with the first electrode and the second electrode on the distal end of the prong.

The method further includes contacting the heart with the first electrode on a first side of the left anterior descending artery of the heart, and contacting the heart with the second electrode on a second side of the left anterior descending artery of the heart.

Wherein the first electrode and the second electrode are positioned on a first side of the left anterior descending artery, and wherein contacting the Manicka Zone of Pacing of the heart with the first electrode and/or the second electrode further includes contacting the Manicka Zone of Pacing of the heart with at least the first electrode.

The method further includes aligning the first electrode and/or the second electrode with a portion of the native conductive pathway of the heart.

The method further includes aligning the first electrode and/or the second electrode with an interventricular septum, a bundle of His, a left bundle branch, and/or a right bundle branch.

Wherein the subcutaneously implantable device further includes a third electrode at a distal end of the prong.

Wherein delivering the pacing signal to the Manicka Zone of Pacing of the heart through the first electrode and/or the second electrode further includes synchronously delivering the pacing signal to the right ventricle and the left ventricle of the heart.

The method further includes delivering the pacing signal to the bundle of His, the left bundle branch, the right bundle branch, and/or the Purkinje fibers.

Wherein contacting the Manicka Zone of Pacing of the heart with the first electrode and/or the second electrode on the distal end of the prong further includes contacting a pericardium or an epicardium of the heart in the Manicka Zone of Pacing of the heart with the first electrode on the distal end of the prong.

A subcutaneously implantable device includes a housing, a clip attached to a top side of the housing that is configured to anchor the device to a xiphoid process and/or a sternum, and a prong with a proximal end attached to the housing and a distal end extending away from the housing that is configured to be positioned at a Manicka Zone of Pacing of a heart. The Manicka Zone of Pacing is defined as an area of the heart extending 3 centimeters laterally outward from either side of a left anterior descending artery of the heart. The device further includes a first electrode on the distal end of the prong that is configured to contact the heart in the Manicka Zone of Pacing, and a second electrode on the distal end of the prong, wherein the second electrode can be configured to contact the heart in the Manicka Zone of Pacing. Circuitry in the housing is in electrical communication with the first electrode and the second electrode and is configured to deliver a pacing signal to the Manicka Zone of Pacing of the heart.

The device of the preceding paragraph can optionally include, additionally and/or alternatively, any one or more of the following features, configurations and/or additional components:

The device further includes a first wire extending through the prong, wherein the first electrode is connected to a distal end of the first wire, and wherein the first wire extends outward from the prong in a first direction; and a second wire extending through the prong, wherein the second electrode is connected to a distal end of the second wire, and wherein the second wire extends outward from the prong in a second direction.

Wherein the first direction is opposite the second direction.

Wherein the first wire and the second wire are stiff wires.

Wherein the first wire exits the prong at a first position and the second wire exits the prong at a second position that is longitudinally spaced from the first position.

Wherein the first wire exits the prong at a side of the prong, and wherein the second wire exits the prong at the distal end of the prong.

Wherein first wire is angled at a 15 to 90 degree angle with respect to a longitudinal axis of the prong, and wherein the second wire is angled at a 15 to 90 degree angle with respect to the longitudinal axis of the prong.

Wherein the first electrode and the second electrode are spaced laterally and longitudinally apart from one another.

Wherein the first electrode and the second electrode are spaced 1.5 to 6 centimeters (0.5906 to 2.3622 inches) from one another.

Wherein the first electrode is positioned 1 to 4 centimeters (0.3937 to 1.5748 inches) from a longitudinal axis of the prong, and wherein the second electrode is positioned 1 to 4 centimeters (0.3937 to 1.5748 inches) from the longitudinal axis of the prong.

A method of pacing a heart includes implanting a pacing device in a patient. The pacing device includes a housing and a first electrode electrically coupled to the housing. The method further includes contacting a Manicka Zone of Pacing on an anterior surface of the heart with the first electrode. The Manicka Zone of Pacing is defined as an area of the heart extending 3 centimeters (1.1811 inches) laterally outward from either side of a left anterior descending artery of the heart. A pacing signal is delivered to the Manicka Zone of Pacing of the heart through the first electrode. A right ventricle and a left ventricle of the heart are synchronously paced with the pacing signal received in the Manicka Zone of Pacing.

The method of the preceding paragraph can optionally include, additionally and/or alternatively, any one or more of the following features, configurations and/or additional components:

Wherein the first electrode contacts the heart in an area extending longitudinally from a region superior to the base of the heart to an apex of the heart within the Manicka Zone of Pacing.

Wherein the first electrode contacts the heart near the base of the heart within the Manicka Zone of Pacing.

The method further includes aligning the first electrode with a portion of the native conductive pathway of the heart.

The method further includes aligning the first electrode contacting the anterior surface of the heart with an interventricular septum, a bundle of His, a left bundle branch, and/or a right bundle branch.

Wherein delivering the pacing signal to the Manicka Zone of Pacing of the heart through the first electrode further includes synchronously delivering the pacing signal to the right ventricle and the left ventricle of the heart.

The method further includes delivering the pacing signal to the bundle of His.

The method further includes delivering the pacing signal to the bundle of His, the left bundle branch, the right bundle branch, and/or the Purkinje fibers.

Wherein contacting the Manicka Zone of Pacing of the heart with the first electrode further includes contacting a pericardium or an epicardium of the heart in the Manicka Zone of Pacing of the heart with the first electrode.

The method further includes actively fixing the first electrode in the myocardial tissue in the Manicka Zone of Pacing of the heart.

A method of pacing a heart includes implanting a pacing device in a patient. The pacing device includes a housing, a first electrode electrically coupled to the housing, and a second electrode electrically coupled to the housing. The method further includes contacting a Manicka Zone of Pacing of the heart with the first electrode and/or the second electrode. The Manicka Zone of Pacing is defined as an area of the heart extending 3 centimeters (1.1811 inches) laterally outward from either side of a left anterior descending artery of the heart, and wherein the first electrode and/or second electrode contact the heart near the base of the heart within the Manicka Zone of Pacing. A pacing signal is delivered to the Manicka Zone of Pacing of the heart through the first electrode and/or the second electrode. A right ventricle and a left ventricle of the heart are synchronously paced with the pacing signal received in the Manicka Zone of Pacing.

The method of the preceding paragraph can optionally include, additionally and/or alternatively, any one or more of the following features, configurations and/or additional components:

Wherein contacting the Manicka Zone of Pacing of the heart with the first electrode and/or the second electrode further includes contacting the Manicka Zone of Pacing of the heart with the first electrode and the second electrode.

The method further includes contacting the heart with the first electrode on a first side of the left anterior descending artery of the heart, and contacting the heart with the second electrode on a second side of the left anterior descending artery of the heart.

Wherein the first electrode and the second electrode are positioned on a first side of the left anterior descending artery, and wherein contacting the Manicka Zone of Pacing of the heart with the first electrode and/or the second electrode further includes contacting the Manicka Zone of Pacing of the heart with at least the first electrode.

The method further includes aligning the first electrode and/or the second electrode with a portion of the native conductive pathway of the heart.

The method further includes aligning the first electrode and/or the second electrode with an interventricular septum, a bundle of His, a left bundle branch, and/or a right bundle branch.

Wherein the subcutaneously implantable device further includes a third electrode electrically coupled to the housing.

Wherein delivering the pacing signal to the Manicka Zone of Pacing of the heart through the first electrode and/or the second electrode further includes synchronously delivering the pacing signal to the right ventricle and the left ventricle of the heart.

The method further includes delivering the pacing signal to the bundle of His, the left bundle branch, the right bundle branch, and/or the Purkinje fibers.

Wherein contacting the Manicka Zone of Pacing of the heart with the first electrode and/or the second electrode further includes contacting a pericardium or an epicardium of the heart in the Manicka Zone of Pacing of the heart with the first electrode and/or the second electrode.

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed.

1. A method of pacing a heart, the method comprising:
implanting a pacing device in a patient, wherein the pacing device includes a housing and a first electrode electrically coupled to the housing;
contacting a Manicka Zone of Pacing on an anterior surface of the heart with the first electrode, wherein the Manicka Zone of Pacing is defined as an area of the heart extending 3 centimeters (1.1811 inches) laterally outward from either side of a left anterior descending artery of the heart;
delivering a pacing signal to the Manicka Zone of Pacing of the heart through the first electrode; and
synchronously pacing a right ventricle and a left ventricle of the heart with the pacing signal received in the Manicka Zone of Pacing.

2. The method of claim 1, wherein the first electrode contacts the heart in an area extending longitudinally from a region superior to the base of the heart to an apex of the heart within the Manicka Zone of Pacing.

3. The method of claim 1, wherein the first electrode contacts the heart near the base of the heart within the Manicka Zone of Pacing.

4. The method of claim 1, and further comprising:
aligning the first electrode with a portion of the native conductive pathway of the heart.

5. The method of claim 1, and further comprising:
aligning the first electrode contacting the anterior surface of the heart with an interventricular septum, a bundle of His, a left bundle branch, and/or a right bundle branch.

6. The method of claim 1, wherein delivering the pacing signal to the Manicka Zone of Pacing of the heart through the first electrode further comprises:
synchronously delivering the pacing signal to the right ventricle and the left ventricle of the heart.

7. The method of claim 1, and further comprising:
delivering the pacing signal to the bundle of His.

8. The method of claim 1, and further comprising:
delivering the pacing signal to the bundle of His, the left bundle branch, the right bundle branch, and/or the Purkinje fibers.

9. The method of claim 1, wherein contacting the Manicka Zone of Pacing of the heart with the first electrode further comprises:
contacting a pericardium or an epicardium of the heart in the Manicka Zone of Pacing of the heart with the first electrode.

10. The method of claim 1, and further comprising:
actively fixing the first electrode in the myocardial tissue in the Manicka Zone of Pacing of the heart.

11. A method of pacing a heart, the method comprising:
implanting a pacing device in a patient, wherein the pacing device includes a housing, a first electrode electrically coupled to the housing, and a second electrode electrically coupled to the housing;
contacting a Manicka Zone of Pacing of the heart with the first electrode and/or the second electrode, wherein the Manicka Zone of Pacing is defined as an area of the heart extending 3 centimeters (1.1811 inches) laterally outward from either side of a left anterior descending artery of the heart, and wherein the first electrode and/or second electrode contact the heart near the base of the heart within the Manicka Zone of Pacing;
delivering a pacing signal to the Manicka Zone of Pacing of the heart through the first electrode and/or the second electrode; and
synchronously pacing a right ventricle and a left ventricle of the heart with the pacing signal received in the Manicka Zone of Pacing.

12. The method of claim 11, wherein contacting the Manicka Zone of Pacing of the heart with the first electrode and/or the second electrode further comprises:
contacting the Manicka Zone of Pacing of the heart with the first electrode and the second electrode.

13. The method of claim 12, and further comprising:
contacting the heart with the first electrode on a first side of the left anterior descending artery of the heart; and
contacting the heart with the second electrode on a second side of the left anterior descending artery of the heart.

14. The method of claim 11, wherein the first electrode and the second electrode are positioned on a first side of the left anterior descending artery, and wherein contacting the Manicka Zone of Pacing of the heart with the first electrode and/or the second electrode further comprises:
contacting the Manicka Zone of Pacing of the heart with at least the first electrode.

15. The method of claim 11, and further comprising:
aligning the first electrode and/or the second electrode with a portion of the native conductive pathway of the heart.

16. The method of claim 11, and further comprising:
aligning the first electrode and/or the second electrode with an interventricular septum, a bundle of His, a left bundle branch, and/or a right bundle branch.

17. The method of claim 11, wherein the subcutaneously implantable device further includes a third electrode electrically coupled to the housing.

18. The method of claim 11, wherein delivering the pacing signal to the Manicka Zone of Pacing of the heart through the first electrode and/or the second electrode further comprises:
synchronously delivering the pacing signal to the right ventricle and the left ventricle of the heart.

19. The method of claim 11, and further comprising:
delivering the pacing signal to the bundle of His, the left bundle branch, the right bundle branch, and/or the Purkinje fibers.

20. The method of claim 11, wherein contacting the Manicka Zone of Pacing of the heart with the first electrode and/or the second electrode further comprises:
contacting a pericardium or an epicardium of the heart in the Manicka Zone of Pacing of the heart with the first electrode and/or the second electrode.
